(19) United States
(12) Patent Application Publication
(10) Pub. No.: US 2011/0275689 A1
(43) Pub. Date: Nov. 10, 2011

(54) Preparation of 3-Pyrrole Substituted 2-Indolinone Derivatives

(75) Inventors: Abhay Gaitonde, Maharashtra (IN); Bharati Choudhari, Maharashtra (IN); Prakash Bansode, Maharashtra (IN); Sunanda Phadtare, Maharashtra (IN)

(73) Assignee: GENERICS [UK] LIMITED, Hertfordshire (GB)

(21) Appl. No.: 13/002,281

(22) PCT Filed: Jul. 1, 2009
(86) PCT No.: PCT/GB09/50771

§ 371 Date: Jul. 25, 2011

(30) Foreign Application Priority Data
Jul. 2, 2008 (IN) 1153/KOL/2008

(51) Int. Cl. A61K 31/404; C07D 403/06; A61P 35/00; C07D 209/34; A61K 31/40; C07D 207/34; C07D 403/04

(52) U.S. Cl. 514/414; 548/531; 548/537; 548/468; 548/466; 548/486; 514/418; 514/423

(57) ABSTRACT

The present invention relates to novel intermediates and further to the use of said intermediates in processes for the preparation of indolinone derivatives, in particular 3-pyrrole substituted 2-indolinones having amide moieties on the pyrrole ring. Such compounds are useful in die treatment of abnormal cell growth, such as cancer, in mammals.

### FIELD OF THE INVENTION

The present invention relates to a novel process for the preparation of indolinone derivatives, in particular 3-pyrrole substituted 2-indolinones having amide moieties on the pyrrole ring. Such compounds are useful in the treatment of abnormal cell growth, such as cancer, in mammals. The invention further relates to novel intermediates useful in said process and to compositions comprising indolinone derivatives as prepared by said process.

### BACKGROUND OF THE INVENTION

Pyrrole substituted indolinone compounds, in particular those having an amide group on the pyrrole ring have been of interest. These compounds modulate protein kinase activity and are thus useful in treating diseases relating to abnormal protein kinase activity, for example various types of cancer.

A process for preparing the amide derivatives is disclosed in WO 01/60814. An appropriate pyrrole is formylated and subsequently condensed with a 2-indolinone to give a respective 5-(2-oxo-1,2-dihydroindole-3-ylidenemethyl)-1H-pyrrole. If a particular amide derivative of the pyrrole is desired, a formylated pyrrole having a carboxylic acid group is selected. The carboxylic acid group is reacted with the desired amine in the presence of DMF, 1-ethyl-3-(3-dimethylaminopropyl)carbodiimide and 1-hydroxybenzotriazole. A scale-up procedure is also disclosed in which the amidation is conducted in the presence of DMF, benzotriazol-1-yloxytris(dimethylamino)phosphonium hexafluorophosphate (BOP) and TEA.

US 2003/0229229 relates to methods of synthesizing pyrrole substituted indolinone compounds having amide moieties on the pyrrole ring. The reaction proceeds via a pyrrole compound having aldehyde and acid moieties at the 5- and 3-positions respectively, which is then coupled with an amine and an oxindole to form the desired pyrrole substituted indolinone compound.

US 2006/0009510 relates to a method of synthesizing indolinone compounds, particularly pyrrole substituted indolinone compounds having amide moieties on the pyrrole ring. The method involves combining 2-oxindole with an amide substituted pyrrole compound in the presence of a formylating agent. This application refers to the process disclosed in US 2003/0229229, stating that the use of an acid-aldehyde substituted pyrrole compound results in consumption of excess amine due to formation of an imine-amide intermediate. This is overcome in the claimed process by utilizing a pyrrole intermediate with the desired amide substitution already in place.

Other examples of such compounds and their synthesis can be found, for example in WO 01/45689, WO 99/48868, U.S. Pat. No. 6,316,429, U.S. Pat. No. 6,316,635, U.S. Pat. No. 6,133,305, U.S. Pat. No. 6,248,771 and GB 1,384,097.

An example of a commercially available pyrrole substituted indolinone is sunitinib malate, marketed as Sutent®. Sunitinib is a multi-targeted receptor tyrosine kinase (RTK) inhibitor that was approved by the FDA for the treatment of renal cell carcinoma (RCC) and imatinib-resistant gastrointestinal stromal tumor (GIST).

In view of the importance of pyrrole substituted indolinones for the treatment of cancer, there is a great need for developing an alternative, relatively simple, economical and commercially feasible process for the synthesis of pyrrole substituted indolinones with a commercially acceptable yield and high purity.

### SUMMARY OF THE INVENTION

It is therefore an object of the present invention to provide a novel improved but simple, economical and commercially feasible process for the synthesis of pyrrole substituted indolinones with a commercially acceptable yield and high purity.

The present inventors have surprisingly found that pyrrole substituted indolinones can be prepared with very high purity employing a simple and efficient process comprising novel intermediates. The prior art processes all employ 2-oxindole as an intermediate which is then coupled with an aldehyde substituted pyrrole compound. The present inventors have surprisingly found that utilizing a novel aldehyde substituted 2-oxindole results in pyrrole substituted indolinones of high purity.

Accordingly, in a first aspect there is provided a process for the preparation of a 3-pyrrole substituted 2-indolinone of formula (I)

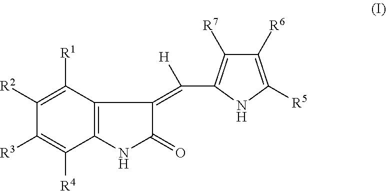

wherein:

R¹ is selected from the group consisting of hydrogen, halo, alkyl, cycloalkyl, aryl, heteroaryl, heteroalicyclic, hydroxy, alkoxy, —C(O)R¹⁵, —NR¹³R¹⁴, —(CH₂)ᵣR¹⁶ and —C(O)NR⁸R⁹;

R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, —S(O)₂NR¹³R¹⁴ and —SO₂R²⁰;

R³ is selected from the group consisting of hydrogen, halo, alkyl, trihalomethyl, hydroxy, alkoxy, —C(O)R¹⁵, —NR¹³R¹⁴, —NR¹³C(O)R¹⁴, —NR¹³C(O)OR¹⁴ and —SO₂R²⁰;

R⁴ is selected from the group consisting of hydrogen, halo, alkyl, hydroxy, alkoxy and —NR¹³R¹⁴;

R⁵ is selected from the group consisting of hydrogen, alkyl and —C(O)R¹⁰;

R⁶ is selected from the group consisting of hydrogen, alkyl and —C(O)R¹⁰;

R⁷ is selected from the group consisting of hydrogen, alkyl, aryl, heteroaryl, —C(O)R¹⁰ and —C(O)R¹⁷; or

R⁶ and R⁷ may combine to form a group selected from the group consisting of —(CH₂)₄—, —(CH₂)₅— and —(CH₂)₆—;

with the proviso that at least one of R⁵, R⁶ or R⁷ must be —C(O)R¹⁰;

R⁸ and R⁹ are independently selected from the group consisting of hydrogen, alkyl and aryl;

R¹⁰ is selected from the group consisting of hydroxy, alkoxy, aryloxy, —N(R¹¹)(CH₂)ₙR¹² and —NR¹³R¹⁴;

R¹¹ is selected from the group consisting of hydrogen and alkyl;

R¹² is selected from the group consisting of —NR¹³R¹⁴, hydroxy, —C(O)R¹⁵, aryl, heteroaryl, —N⁺(O⁻)R¹³R¹⁴, —N(OH)R¹³ and —NHC(O)R^{a} (wherein R^{a} is unsubstituted alkyl, haloalkyl or aralkyl);

R¹³ and R¹⁴ are independently selected from the group consisting of hydrogen, alkyl, cyanoalkyl, cycloalkyl, aryl and heteroaryl; or

R¹³ and R¹⁴ may combine to form a heterocycle group;

R¹⁵ is selected from the group consisting of hydrogen, alkoxy, hydroxy and aryloxy;

R¹⁶ is selected from the group consisting of hydroxy, —C(O)R¹⁵, —NR¹³R¹⁴ and —C(O)NR¹³R¹⁴;

R¹⁷ is selected from the group consisting of alkyl, cycloalkyl, aryl and heteroaryl;

R²⁰ is alkyl, aryl, aralkyl, heteroaryl or heteroaralkyl; and

n and r are independently 1, 2, 3 or 4;

or a salt such as a pharmaceutically acceptable salt thereof;

comprising the step of reacting a compound of formula (III)

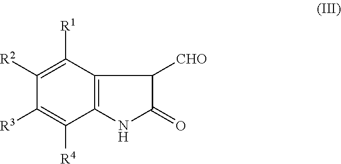

or a salt thereof, wherein R¹ to R⁴ are as hereinbefore described, with a compound of formula (II)

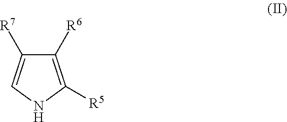

or a salt thereof, wherein R⁵ to R⁷ are as hereinbefore described.

In a preferred embodiment of the first aspect of the present invention, R¹, R², R³ and R⁴ are each independently selected from hydrogen or a fluoro, chloro or bromo group. More preferably R¹, R³ and R⁴ are each hydrogen and R² is selected from a fluoro, chloro or bromo group. Most preferably R¹, R³ and R⁴ are each hydrogen and R² is a fluoro group.

In another embodiment of the first aspect of the present invention, R²⁰ is alkyl, aryl, aralkyl or heteroaryl.

In one embodiment of the process, at least one of R⁵, R⁶ and R⁷ is —COOH. Preferably one of R⁵, R⁶ and R⁷ is —COOH and two of R⁵, R⁶ and R⁷ are independently selected from hydrogen or an alkyl group such as a C₁₋₄ alkyl group. Preferably any alkyl groups of R⁵, R⁶ and R⁷ are unsubstituted. Preferably R⁶ is —COOH. Preferably compound (II) is a carboxylic acid having structure (IIa)

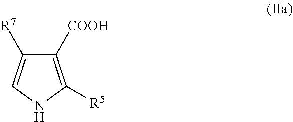

or a salt thereof, preferably wherein R⁵ and R⁷ are independently selected from hydrogen or an alkyl group such as a C₁₋₄ alkyl group, more preferably wherein R⁵ and R⁷ are independently selected from a C₁₋₄ alkyl group, and most preferably wherein R⁵ and R⁷ are methyl.

In an alternative process according to the invention, at least one of R⁵, R⁶ and R⁷ is —COR wherein R is selected from the group consisting of —N(R¹¹)(CH₂)ₙR¹² and —NR¹³R¹⁴; and R¹¹ to R¹⁴ and n are as hereinbefore described. Preferably one of R⁵, R⁶ and R⁷ is —COR and two of R⁵, R⁶ and R⁷ are independently selected from hydrogen or an alkyl group such as a C₁₋₄ alkyl group. More preferably one of R⁵, R⁶ and R⁷ is —COR and two of R⁵, R⁶ and R⁷ are independently selected from a C₁₋₄ alkyl group. Preferably any alkyl groups of R⁵, R⁶ and R⁷ are unsubstituted. Preferably R⁶ is —COR. Preferably compound (II) is an amide having structure (IIb)

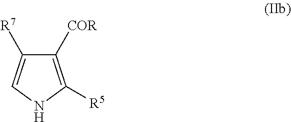

or a salt thereof, wherein:

R⁵ and R⁷ are as hereinbefore described;

R is selected from the group consisting of —N(R¹¹)(CH₂)ₙR¹² and —NR¹³R¹⁴; and

R¹¹ to R¹⁴ and n are as hereinbefore described.

Most preferably R⁵ and R⁷ are methyl and/or R is —NH(CH₂)₂NEt₂.

In a preferred embodiment of a process according to the first aspect of the invention, the reaction occurs in an acidified polar solvent system. The polar solvent may be selected from polar aprotic solvents including ethers such as THF (tetrahydrofuran), diethyl ether and methyl t-butyl ether, N,N-dimethylformamide, dimethylsulfoxide, acetonitrile, esters such as ethyl acetate, and ketones such as acetone. Preferably the solvent is a polar protic solvent such as an alcohol or a carboxylic acid. More preferably the solvent is a hydroxylic organic solvent, preferably an alcohol. Preferably the alcohol is R^{α}OH, wherein R^{α} is selected from an optionally substituted alkyl or aralkyl group. Preferably the alcohol is monohydric. Preferably R^{α} is an optionally substituted C₁₋₈ alkyl group, more preferably R^{α} is an optionally substituted C₁₋₄ alkyl group. Preferably the alcohol is methanol, ethanol, 1-propanol, isopropanol, 1-butanol, 2-methyl-1-propanol, t-butanol, 1-pentanol, cyclopentanol, 1-hexanol, cyclohexanol, 1-heptanol or 1-octanol. Most preferably the solvent is ethanol.

In an alternate embodiment of a process according to the first aspect of the invention, the reaction occurs in an acidified non-polar solvent system, such as acidified toluene.

In another embodiment the acid is selected from the group comprising hydrohalogenic acids (for example, hydrofluoric, hydrochloric, hydrobromic or hydroiodic acid) or other mineral acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). Preferably the acid is selected from hydrohalogenic and other mineral acids, for example, hydrochloric acid, concentrated hydrochloric acid, sulfuric acid, concentrated sulfuric acid, and organic acids such as glacial acetic acid, p-toluene sulfonic acid. More preferably the acid is a hydrohalogenic acid. Most preferably the acid is hydrochloric acid, in particular when the solvent is ethanol.

In one embodiment of the first aspect of the present invention, the reaction occurs at a temperature of from 20 to 200° C., more preferably at a temperature of from 50 to 150° C., more preferably still at a temperature of from 70 to 100° C., most preferably at a temperature of about 80° C. In one embodiment, the reaction occurs at the reflux temperature of the solvent.

Preferably the reaction of the first aspect of the present invention occurs over a period of 30 minutes to 48 hours. More preferably the reaction occurs over a period of 2 to 24 hours, more preferably still over a period of 4 to 18 hours. Most preferably the reaction occurs over a period of 6 to 12 hours.

The inventors have found that utilizing the novel intermediate having structure (III) and in particular intermediate (IIIa) in the preparation of sunitinib results in a process that provides the claimed advantages. This intermediate is not taught or even alluded to in the prior art documents where it is only the 2-oxindole intermediate without the aldehyde substitution at the 3-position of the indole ring that is taught.

One embodiment of the first aspect according to the invention provides a process for preparing sunitinib having structure:

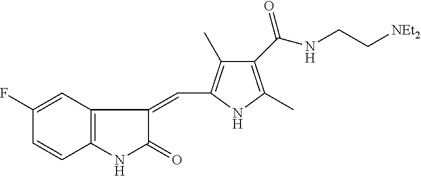

or a pharmaceutically acceptable salt thereof, the process comprising the step of reacting a compound of formula (IIc)

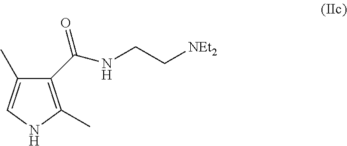

or a salt thereof, with a compound of formula (IIa)

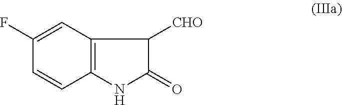

or a salt thereof.

In an alternative embodiment according to the first aspect according to the invention, a process for preparing sunitinib having structure

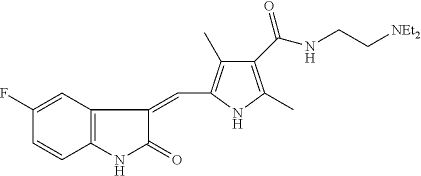

or a pharmaceutically acceptable salt thereof, is provided, comprising the steps of reacting a compound of formula (IIIa)

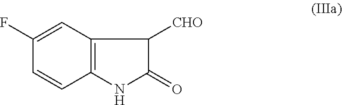

or a salt thereof, with a compound of formula (IIa) or (IIb)

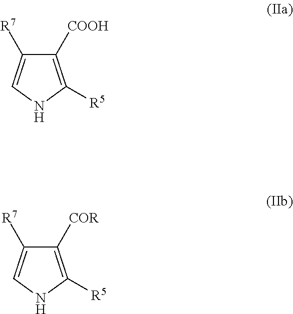

or a salt thereof, wherein R⁵ and R⁷ are both methyl groups and R is as hereinbefore described, and optionally converting the resulting intermediate to sunitinib.

Preferably according to either of the above two embodiments of the first aspect of the invention, a process is provided wherein the reaction occurs in an acidified polar solvent system, such as one discussed above. Preferably the solvent is a hydroxylic organic solvent, most preferably the solvent system is ethanolic hydrogen chloride.

Preferably also according to either of the above two embodiments, the acid is selected from those discussed above in relation to the first aspect of the present invention, more preferably the acid is selected from the group comprising mineral acids, for example, hydrochloric acid, concentrated hydrochloric acid, sulfuric acid, concentrated sulfuric acid, and organic acids such as glacial acetic acid, p-toluene sulfonic acid. Preferably the acid is hydrochloric acid, in particular when the solvent is ethanol.

A second aspect of the present invention provides a process for preparing an acid of formula (IIa), (IIa′) or (IIa″)

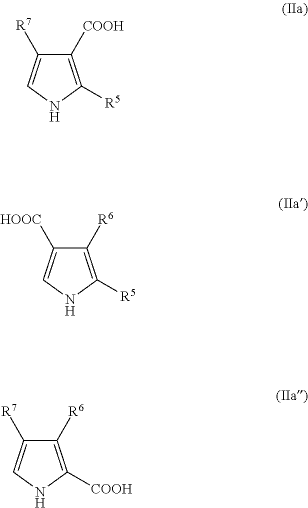

or a salt thereof, wherein said acid (IIa), (IIa′) or (IIa″) is formed from the corresponding pyrrole ester (IId), (IId′) or (IId″)

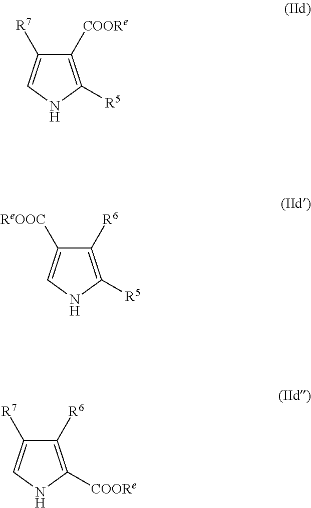

or a salt thereof, wherein;

R⁵ to R⁷ are as hereinbefore described; and

R^{e} is an alkyl, aryl, heteroaryl, aralkyl, cycloalkyl or heterocycle group.

The acid (IIa), (IIa′) or (IIa″) may be formed from the corresponding pyrrole ester (IId), (IId′) or (IId″) by any method known in the art, such as those exemplified in “Protective Groups in Organic Synthesis” by T. W. Greene and P. G. M. Wuts (Wiley-Interscience, 4^{th} edition, 2006). For instance where R^{e} is an aralkyl group such as a benzyl group, the acid may be formed from the corresponding pyrrole ester by hydrogenation.

Preferably the acid (IIa), (IIa′) or (IIa″), or a salt thereof, is formed from the corresponding pyrrole ester (IId), (IId′) or (IId″), or a salt thereof, by hydrolysis.

In those aspects and embodiments that employ the pyrrole intermediate (IIa), there is provided a preferred embodiment of the second aspect according to the invention comprising a process wherein the acid (IIa) or a salt thereof is formed by hydrolysis of pyrrole ester (IId)

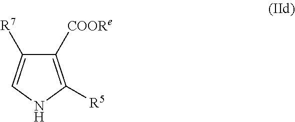

or a salt thereof, wherein:

R⁵ and R⁷ are as hereinbefore described; and

R^{e} is an alkyl, aryl, heteroaryl, aralkyl, cycloalkyl or heterocycle group.

The hydrolysis of the second aspect of the invention may be acid or base catalysed. Preferably the hydrolysis is base catalysed. In certain embodiments according to the invention, the hydrolysis is performed in a solvent system comprising one or more polar solvent(s) and a base. The polar solvent(s) may be selected from polar aprotic solvents including N,N-dimethylformamide, dimethylsulfoxide, acetonitrile and ketones such as acetone, or from polar protic solvents including water, alcohols, carboxylic acids and amines, or from mixtures thereof. Preferably the solvent system comprises water, optionally with a second polar protic solvent such as an alcohol.

Preferably the solvent system comprises 1 to 50% water by volume, more preferably 5 to 25% water by volume, most preferably 10 to 15% water by volume.

Where an alcohol is used, preferably the alcohol is R^{β}OH, wherein R^{β} is selected from an optionally substituted alkyl or aralkyl group. Preferably the alcohol is monohydric. Preferably R^{β} is an optionally substituted C₁₋₈ alkyl group, more preferably R^{β} is an optionally substituted C₁₋₄ alkyl group. Preferably the alcohol is methanol, ethanol, 1-propanol, isopropanol, 1-butanol, 2-methyl-1-propanol, t-butanol, 1-pentanol, cyclopentanol, 1-hexanol, cyclohexanol, 1-heptanol or 1-octanol. Most preferably the alcohol is methanol.

Preferably the base is an alkoxide base such as a methoxide, ethoxide, t-butoxide, or an aryloxide base such as a phenoxide, or a hydroxide base. More preferably the base is a hydroxide base, preferably an alkali metal hydroxide such as sodium or potassium hydroxide.

In one embodiment of the second aspect of the present invention, the solvent system is a combination of methanol and potassium hydroxide. Alternatively, the solvent comprises one or more of the group comprising water, one or more alcohols and a base. In a preferred embodiment the solvent comprises a combination of water and methanol, which in a particularly preferred embodiment are in a ratio of about 0.4:3. In another embodiment of the process according to the second aspect, the base is an inorganic base. In a particularly preferred embodiment, the inorganic base is potassium hydroxide. The inventors have found a solvent system comprising methanol, water and potassium hydroxide to be particularly advantageous, in particular in the preparation of sunitinib.

Preferably the hydrolysis of the second aspect of the present invention occurs at a temperature of from 20 to 200° C., more preferably at a temperature of from 50 to 150° C., more preferably still at a temperature of from 60 to 110° C., most preferably at a temperature of about 65° C. In one embodiment, the reaction occurs at the reflux temperature of the solvent system.

Preferably the hydrolysis of the second aspect of the present invention occurs over a period of 30 minutes to 48 hours. More preferably the hydrolysis occurs over a period of 1 to 24 hours, more preferably still over a period of 3 to 12 hours. Most preferably the hydrolysis occurs over a period of 5 to 6 hours.

In another embodiment of the second aspect of the present invention, R^{e} is an alkyl or cycloalkyl group. Preferably R^{e} comprises from 1 to 6 carbon atoms, more preferably from 1 to 4 carbon atoms. More preferably R^{e} is selected from a methyl, ethyl, iso-propyl or n-propyl group. Most preferably R^{e} is an ethyl group.

A particularly preferred embodiment of the second aspect provides a process wherein the pyrrole ester (IId) is a compound having structure (IIe)

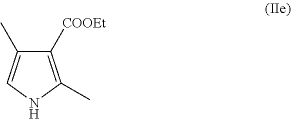

or a salt thereof.

A third aspect of the present invention provides a process for preparing an amide of formula (IIb), (IIb′) or (IIb″)

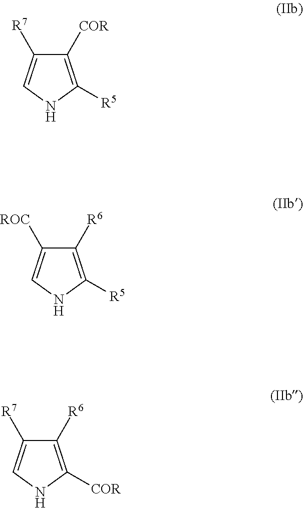

or a salt thereof, wherein said amide (IIb), (IIb′) or (IIb″) is formed from the corresponding acid (IIa), (IIa′) or (IIa″)

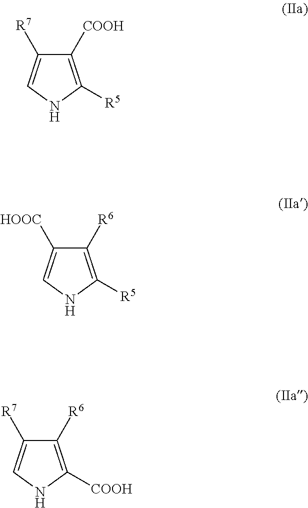

or a salt thereof, wherein R and R⁵ to R⁷ are as hereinbefore described.

Preferably said process is for preparing an amide of formula (IIb) or a salt thereof, from the corresponding acid (IIa) or a salt thereof. More preferably said process is for preparing the amide (IIc)

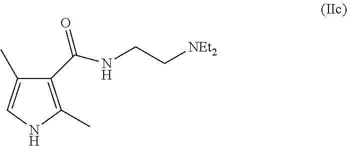

or a salt thereof, from the corresponding acid (IIf)

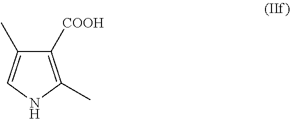

or a salt thereof.

A fourth aspect of the present invention relates to a process for preparing an amide of formula (Ib), (Ib′) or (Ib″)

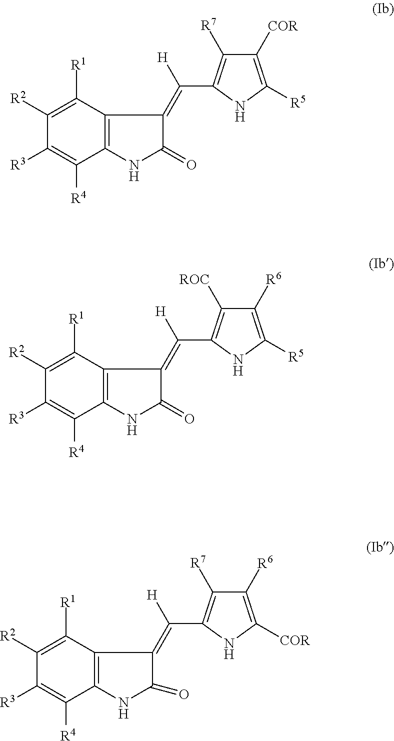

or a salt such as a pharmaceutically acceptable salt thereof, wherein said amide (Ib), (Ib′) or (Ib″) is formed from the corresponding acid (Ia), (Ia′) or (Ia″)

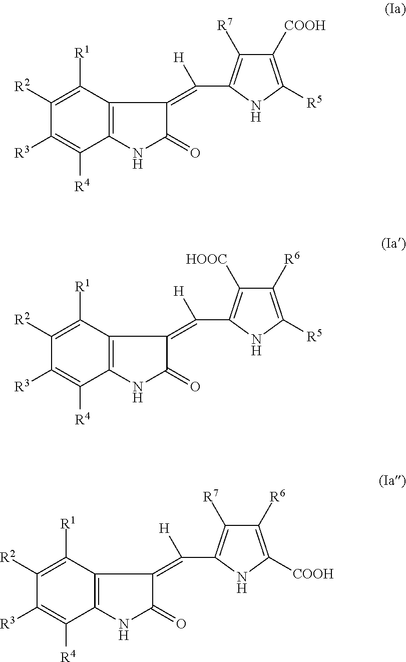

or a salt thereof, wherein R and R¹ to R⁷ are as hereinbefore described.

Preferably said process is for preparing an amide of formula (Ib) or a salt thereof, from the corresponding acid (Ia) or a salt thereof. More preferably said process is for preparing sunitinib having structure:

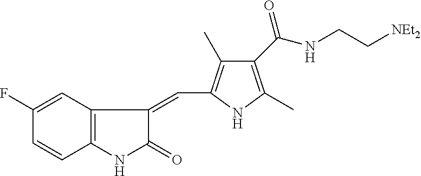

or a salt such as a pharmaceutically acceptable salt thereof, from the corresponding acid 5-[(5-fluoro-2-oxo-1,2-dihydro-3H-indol-3-ylidene)methyl]-2,4-dimethyl-1H-pyrrole-3-carboxylic acid:

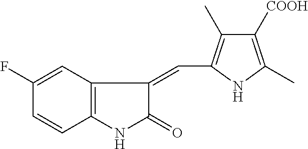

or a salt thereof.

In one embodiment of either the third or fourth aspects of the present invention, the acid is converted to the corresponding amide via chemical activation of the —COOH group and subsequent reaction with RH, or a salt thereof, wherein R is as hereinbefore described.

As used herein, “chemical activation” of the —COOH group refers to the use of chemical reagents to convert the —COOH group into a species that is more reactive towards nucleophilic attack, for example, by primary or secondary amines. Methods of performing such chemical activation are well known in the art and include for instance the conversion of the —COOH group into an acyl halide such as —COCl, into an anhydride such as —C(O)OC(O)OMe, or into an active ester such as a pentafluorophenyl ester (—COOPfp), or the use of coupling reagents such as DCC(N,N′-dicyclohexylcarbodiimide) and HOBT (1-hydroxybenzotriazole), TBTU (O-(benzotriazol-1-yl)-N,N,N′,N′-tetramethyluronium tetrafluoroborate or the guanidinium N-oxide isomer thereof) or HAM (O-(7-azabenzotriazol-1-yl)-N,N,N′,N′-tetramethyluronium hexafluorophosphate or the guanidinium N-oxide isomer thereof).

Preferably the chemical activation is achieved via the use of a carbodiimide coupling reagent, optionally in conjunction with 1-hydroxybenzotriazole (HOBT) and/or a suitable base (i.e. one that will not form a side product by reaction with the activated —COOH group) such as a tertiary amine.

Suitable carbodiimide coupling reagents include for instance DCC (N,N′-dicyclohexylcarbodiimide), DIC (N,N′-diisopropylcarbodiimide), EDC (1-ethyl-3-(3-dimethylaminopropyl)carbodiimide) and salts thereof.

Most preferably the chemical activation is achieved via the use of 1-ethyl-3-(3-dimethylaminopropyl)carbodiimide hydrochloride, HOBT and triethylamine (TEA).

Preferably RH is N,N-diethylethylenediamine or a salt thereof.

Preferably from 1 to 10 molar equivalents of RH are used, more preferably from 2 to 5 molar equivalents of RH are used, most preferably about 3 molar equivalents of RH are used.

In one embodiment, the chemical activation and subsequent reaction with RH is performed in an aprotic solvent, preferably a polar aprotic solvent. Suitable polar aprotic solvents include ethers such as THF (tetrahydrofuran), diethyl ether and methyl t-butyl ether, DMF (N,N-dimethylformamide), DMSO (dimethylsulfoxide), acetonitrile, esters such as ethyl acetate, and ketones such as acetone. Most preferably the polar aprotic solvent is THF.

In one embodiment, the chemical activation and subsequent reaction with RH is performed at a temperature of from 0 to 100° C., more preferably at a temperature of from 10 to 50° C., most preferably at a temperature of from 20 to 30° C.

Preferably the reaction with RH occurs over a period of 1 to 48 hours. More preferably the reaction occurs over a period of 3 to 24 hours, more preferably still over a period of 6 to 12 hours. Most preferably the reaction occurs over a period of 8 to 10 hours.

A fifth aspect of the present invention provides a process for preparing a compound of formula (III)

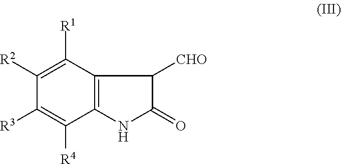

or a salt thereof, comprising adding a formyl group at the 3-position of a 2-oxindole having structure (IIIc)

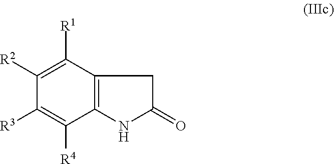

or a salt thereof, wherein R¹ to R⁴ are as hereinbefore described.

In one embodiment of the fifth aspect of the present invention, the process is for preparing a compound of formula (Ma) or a salt thereof according to the invention, comprising adding a formyl group at the 3-position of 5-fluoro-2-oxindole (IIIe)

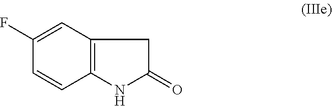

or a salt thereof.

The formyl group may be added using for instance formate esters such as methyl, ethyl, n-propyl or iso-propyl formate; mixed anhydrides of formic acid such as acetic formic anhydride or formic benzenesulfonic anhydride; disubstituted formamides such as N-phenyl-N-methyl-formamide in conjunction with phosphorus oxychloride or phosgene (the Vilsmeier-Haack reaction); chloroform in conjunction with a hydroxide source (the Reimer-Tiemann reaction); dichloromethyl methyl ether in conjunction with AlCl₃; or formyl fluoride and BF₃.

Preferably a formate ester is used. Most preferably the process of the fifth aspect of the present invention comprises reacting 2-oxindole (IIIc) such as 5-fluoro-2-oxindole (IIIe) or a salt thereof with ethyl formate.

In one embodiment of the fifth aspect of the present invention, the formylation is base catalysed. Preferably the base is an alkoxide base such as a methoxide, ethoxide or t-butoxide, or an aryloxide base such as a phenoxide, or an alkali metal such as sodium. More preferably the base is an alkoxide base, preferably a C₁₋₄ alkoxide base such as sodium methoxide or sodium ethoxide.

In another embodiment of the fifth aspect of the present invention, the formylation is performed in a polar solvent. The polar solvent may be selected from polar aprotic solvents including N,N-dimethylformamide, dimethylsulfoxide, acetonitrile, esters such as ethyl acetate, and ketones such as acetone; or from polar protic solvents including alcohols, carboxylic acids and amines; or from mixtures thereof. Preferably the solvent is a polar protic solvent, more preferably a hydroxylic solvent and most preferably the solvent is an alcohol.

Where an alcohol is used, preferably the alcohol is R^{γ}OH, wherein R^{γ} is selected from an optionally substituted alkyl or aralkyl group. Preferably the alcohol is monohydric. Preferably R^{γ} is an optionally substituted C₁₋₈ alkyl group, more preferably R^{γ} is an optionally substituted C₁₋₄ alkyl group. Preferably the alcohol is methanol, ethanol, 1-propanol, isopropanol, 1-butanol, 2-methyl-1-propanol, t-butanol, 1-pentanol, cyclopentanol, 1-hexanol, cyclohexanol, 1-heptanol or 1-octanol. Most preferably the alcohol is methanol.

In certain embodiments of the process, the reaction takes place in the presence of a hydroxylic solvent and one of sodium methoxide, sodium ethoxide or sodium metal.

Preferably the formylation of the fifth aspect of the present invention occurs at a temperature of from 20 to 200° C., more preferably at a temperature of from 50 to 150° C., more preferably still at a temperature of from 60 to 110° C., most preferably at a temperature of about 65° C. In one embodiment, the reaction occurs at the reflux temperature of the solvent.

Preferably the formylation of the fifth aspect of the present invention occurs over a period of 10 minutes to 6 hours. More preferably the formylation occurs over a period of 15 minutes to 3 hours, more preferably still over a period of 30 minutes to 2 hours. Most preferably the formylation occurs over a period of about 1 hour.

In a sixth aspect of the present invention a process for preparing a 2-oxindole compound (IIIc)

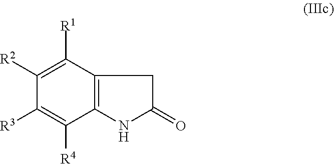

or a salt thereof, is provided, the process comprising reacting hydrazine hydrate with an isatin having structure (IIId)

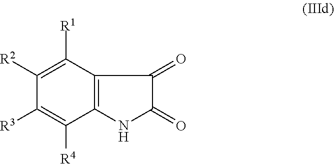

or a salt thereof, wherein R¹ to R⁴ are as hereinbefore described.

Preferably the reaction takes place in the presence of a hydroxylic solvent and one of sodium methoxide, sodium ethoxide or sodium metal.

One embodiment of the sixth aspect according to the invention provides a process for preparing a compound (IIIe) or a salt thereof for use in the synthesis of sunitinib and salts, solvates and crystalline forms thereof, comprising reacting hydrazine hydrate with 5-fluoro-isatin having structure (IIIf)

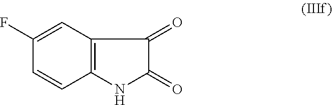

or a salt thereof.

Preferably the reaction takes place in the presence of a hydroxylic solvent and one of sodium methoxide, sodium ethoxide or sodium metal, most preferably in the presence of sodium methoxide. In a particularly preferred embodiment the 5-fluoro-isatin (IIIf) is added stepwise to the hydrazine hydrate.

A seventh aspect of the present invention relates to a method comprising two or more processes selected from:
(a) the process according to the sixth aspect of the present invention;
(b) the process according to the fifth aspect of the present invention;
(c) the process according to the second aspect of the present invention; and
(d) the process according to the first aspect of the present invention.

Optionally the method of the seventh aspect of the present invention comprises three or preferably all four of processes (a) to (d). Preferably the method comprises process (d). In one embodiment, the method of the seventh aspect of the present invention comprises processes (b) and (d). In another embodiment, the method comprises processes (c) and (d), or (b), (c) and (d). In yet another embodiment, the method comprises processes (a), (b) and (d).

Optionally, the two or more processes may further be selected from, or the method of the seventh aspect may include, (e) the process according to the fourth aspect of the present invention. In such a case it is preferred that the method comprises process (d) wherein in the first aspect of the present invention at least one of R⁵, R⁶ and R⁷ is —COOH.

Alternatively, the two or more processes may further be selected from, or the method of the seventh aspect may include, (f) the process according to the third aspect of the present invention. In such a case it is preferred that the method comprises process (d) wherein in the first aspect of the present invention at least one of R⁵, R⁶ and R⁷ is —COR.

An eighth aspect provides a method or process according to any aspect or embodiment according to the invention for the preparation of sunitinib and/or any salt, solvate or polymorph thereof. In a preferred embodiment, the method or process further comprises preparing the malic acid salt of sunitinib. In a particularly preferred embodiment, the malic acid salt is the L-malic acid salt.

A ninth aspect according to the invention provides a compound having structure (III)

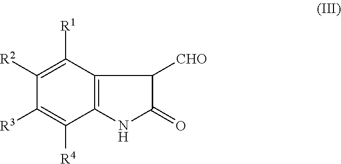

or a salt thereof, wherein R¹ to R⁴ are as hereinbefore described.

As mentioned previously this intermediate is useful in the preparation of pyrrole substituted indolinone compounds. Further, the intermediate is not known from the prior art where reactions between the pyrrole and indolinone intermediates were facilitated by the aldehyde group being present on the pyrrole intermediate.

In a particularly preferred embodiment, there is provided a compound having structure (IIIa)

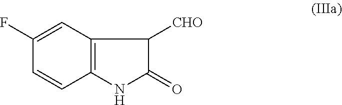

or a salt thereof.

Compound (IIIa) is particularly useful in the preparation of sunitinib.

A tenth aspect according to the invention provides a compound having structure (IIa), (IIa′) or (IIa″)

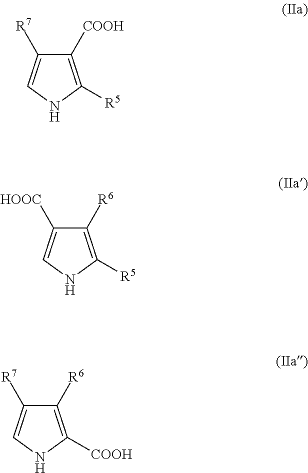

or a salt thereof, wherein. R⁵ to R⁷ are as hereinbefore described.

In one embodiment of the tenth aspect of the present invention, the compound has structure (IIa) or is a salt thereof.

In another embodiment of the tenth aspect of the present invention, R⁵ to R⁷ are each independently selected from hydrogen or alkyl. Preferably R⁵ to R⁷ are each independently selected from hydrogen or C₁₋₄ alkyl. More preferably R⁵ to R⁷ are each independently selected from C₁₋₄ alkyl. Preferably any alkyl groups of R⁵, R⁶ and R⁷ are unsubstituted. Most preferably R⁵ to R⁷ are methyl.

In a preferred embodiment according to the tenth aspect of the present invention, there is provided a compound having structure (IIf)

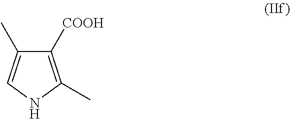

or a salt thereof.

An eleventh aspect according to the invention provides a compound having structure (Ia)

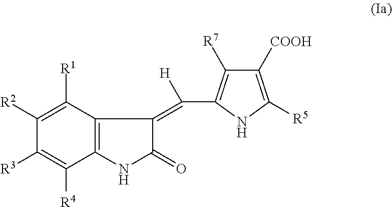

or a salt thereof, wherein:

R¹ to R⁴ are as hereinbefore described; and

R⁵ and R⁷ are each independently selected from hydrogen or alkyl.

In one embodiment of the eleventh aspect of the present invention, R⁵ and R⁷ are each independently selected from hydrogen or C₁₋₄ alkyl. Preferably R⁵ and R⁷ are each independently selected from C₁₋₄ alkyl. Preferably any alkyl groups of R⁵ and R⁷ are unsubstituted. Most preferably R⁵ and R⁷ are methyl.

In a preferred embodiment according to the eleventh aspect of the present invention, there is provided a compound having structure:

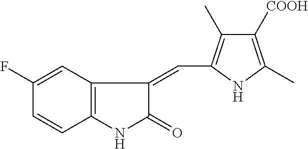

or a salt thereof.

A twelfth aspect according to the present invention relates to a compound of formula (I) or a salt such as a pharmaceutically acceptable salt thereof as prepared according to any of the first eight aspects of the present invention or a compound of formula (I) or a salt such as a pharmaceutically acceptable salt thereof prepared utilising a compound according to any of the ninth, tenth or eleventh aspects of the present invention. Preferably the compound of formula (I) is sunitinib or a pharmaceutically acceptable salt thereof. More preferably the compound of formula (I) is sunitinib malate.

A thirteenth aspect of the present invention provides a pharmaceutical composition comprising a compound of formula (I) or a pharmaceutically acceptable salt thereof according to the twelfth aspect of the present invention and one or more pharmaceutically acceptable excipient(s).

In a particularly preferred embodiment of said composition, the compound is sunitinib malate.

Preferably the composition is a solid oral composition, most preferably a tablet or a capsule, most preferably a tablet.

A fourteenth aspect provides the use of a compound of formula (I) or a pharmaceutically acceptable salt thereof according to the twelfth aspect of the present invention, or of a pharmaceutical composition according to the thirteenth aspect of the present invention, in the treatment of a protein kinase mediated disorder. Preferably the disorder is a cell proliferative disorder, most preferably cancer, particularly preferred is wherein the disorder is a solid tumour, most preferably the disorder is one of advanced renal cell carcinoma (RCC) or gastrointestinal stromal tumor (GIST).

A fifteenth aspect of the present invention provides the use of a compound of formula (I) or a pharmaceutically acceptable salt thereof according to the twelfth aspect of the present invention, or of a pharmaceutical composition according to the thirteenth aspect of the present invention, in the manufacture of a medicament for the treatment of a protein kinase mediated disorder. Preferably the disorder is a cell proliferative disorder, most preferably cancer, particularly preferred is wherein the disorder is a solid tumour, most preferably the disorder is one of advanced renal cell carcinoma (RCC) or gastrointestinal stromal tumor (GIST).

A sixteenth aspect of the present invention provides a method of treating a protein kinase mediated disorder, comprising administering to a patient in need thereof a therapeutically effective amount of a compound of formula (I) or a pharmaceutically acceptable salt thereof according to the twelfth aspect of the present invention, or of a pharmaceutical composition according to the thirteenth aspect of the present invention. Preferably the disorder is a cell proliferative disorder, most preferably cancer, particularly preferred is wherein the disorder is a solid tumour, most preferably the disorder is one of advanced renal cell carcinoma (RCC) or gastrointestinal stromal tumor (GIST). Preferably the patient is a mammal, preferably a human.

For the avoidance of doubt, insofar as is practicable any embodiment of a given aspect of the present invention may occur in combination with any other embodiment of the same aspect of the present invention. In addition, insofar as is practicable it is to be understood that any preferred or optional embodiment of any aspect of the present invention should also be considered as a preferred or optional embodiment of any other aspect of the present invention.

### DETAILED DESCRIPTION OF THE INVENTION

The term “pyrrole substituted indolinones” as used herein throughout the description and claims includes any salt, solvate or polymorph thereof.

For the purposes of the present invention, an “alkyl” group is defined as a saturated aliphatic hydrocarbon radical including straight chain and branched chain groups of 1-20 carbon atoms. Wherever a numerical range, e.g. 1-20, is stated herein, it means that the group, in this case the alkyl group, may contain 1 carbon atom, 2 carbon atoms, 3 carbon atoms, etc. up to and including 20 carbon atoms. Alkyl groups containing 1-4 carbon atoms are referred to as lower alkyl groups. When said lower alkyl groups lack substituents, they are referred to as unsubstituted lower alkyl groups. More preferably, an alkyl group is a medium size alkyl group having 1-10 carbon atoms, e.g. methyl, ethyl, propyl, 2-propyl, n-butyl, iso-butyl, tert-butyl, pentyl and the like. More preferably it is a lower alkyl group having 1-4 carbon atoms, e.g. methyl, ethyl, propyl, 2-propyl, n-butyl, iso-butyl, tert-butyl and the like. The alkyl group may be substituted or unsubstituted.

When substituted, the substituent group(s) is/are preferably one or more, more preferably one to three groups which are independently of each other hydroxy; halo; unsubstituted lower alkyl; unsubstituted lower alkoxy; aryloxy optionally substituted with one or more groups, preferably one, two or three groups, which are independently of each other hydroxy, halo, unsubstituted lower alkyl or unsubstituted lower alkoxy groups; 6-membered heteroaryl having from 1 to 3 nitrogen atoms in the ring, the carbon atoms in the ring being optionally substituted with one or more groups, preferably one, two or three groups, which are independently of each other hydroxy, halo, unsubstituted lower alkyl or unsubstituted lower alkoxy groups; 5-membered heteroaryl having from 1 to 3 heteroatoms in the ring, selected from the group consisting of nitrogen, oxygen and sulfur, the carbon and the nitrogen (if present) atoms in the ring being optionally substituted with one or more groups, preferably one, two or three groups, which are independently of each other hydroxy, halo, unsubstituted lower alkyl or unsubstituted lower alkoxy groups; mercapto; (unsubstituted lower alkyl)thio; arylthio optionally substituted with one or more groups, preferably one, two or three groups, which are independently of each other hydroxy, halo, unsubstituted lower alkyl or unsubstituted lower alkoxy groups; cyano; acyl; thioacyl; O-carbamyl; N-carbamyl; O-thiocarbamyl; N-thiocarbamyl; C-amido; N-amido; nitro; N-sulfonamido; S-sulfonamido; —S(O)R¹⁸; —S(O)₂R¹⁸; —C(O)OR¹⁸; —OC(O)R¹⁸; and —NR¹⁸R¹⁹; wherein R¹⁸ and R¹⁹ are independently selected from the group consisting of hydrogen, unsubstituted lower alkyl, trihalomethyl, unsubstituted (C₃-C₆)cycloalkyl, unsubstituted lower alkenyl, unsubstituted lower alkynyl and aryl optionally substituted with one or more groups, preferably one, two or three groups, which are independently of each other hydroxy, halo, unsubstituted lower alkyl or unsubstituted lower alkoxy groups.

Preferably, the alkyl group is substituted with one or two substituents independently selected from the group consisting of hydroxy; a 5- or 6-membered heteroalicyclic group having from 1 to 3 heteroatoms in the ring, selected from the group consisting of nitrogen, oxygen and sulfur, the carbon and the nitrogen (if present) atoms in the ring being optionally substituted with one or more groups, preferably one, two or three groups, which are independently of each other hydroxy, halo, unsubstituted lower alkyl or unsubstituted lower alkoxy groups; 5-membered heteroaryl having from 1 to 3 heteroatoms in the ring, selected from the group consisting of nitrogen, oxygen and sulfur, the carbon and the nitrogen (if present) atoms in the ring being optionally substituted with one or more groups, preferably one, two or three groups, which are independently of each other hydroxy, halo, unsubstituted lower alkyl or unsubstituted lower alkoxy groups; 6-membered heteroaryl having from 1 to 3 nitrogen atoms in the ring, the carbon atoms in the ring being optionally substituted with one or more groups, preferably one, two or three groups, which are independently of each other hydroxy, halo, unsubstituted lower alkyl or unsubstituted lower alkoxy groups; or —NR¹⁸R¹⁹ wherein R¹⁸ and R¹⁹ are independently selected from the group consisting of hydrogen and unsubstituted lower alkyl.

Even more preferably, the alkyl group is substituted with one or more substituents which are independently of each other hydroxy, dimethylamino, ethylamino, diethylamino, dipropylamino, pyrrolidino, piperidino, morpholino, piperazino, 4-lower alkyl-piperazino, phenyl, imidazolyl, pyridinyl, pyridazinyl, pyrimidinyl, oxazolyl, triazinyl and the like.

“Cycloalkyl” refers to an all-carbon 3- to 8-membered monocyclic ring, such as an all-carbon 5- or 6-membered monocyclic ring, or an all-carbon 6- to 12-membered fused bicyclic ring, or an all-carbon fused polycyclic ring (a “fused” ring system means that each ring in the system shares at least two atoms such as an adjacent pair of atoms with another ring in the system) wherein one or more of the rings may contain one or more double bonds but none of the rings has a completely conjugated pi-electron system. Examples, without limitation, of cycloalkyl groups are cyclopropane, cyclobutane, cyclopentane, cyclopentene, cyclohexane, cyclohexadiene, adamantane, cycloheptane, cycloheptatriene and the like. A cycloalkyl group may be substituted or unsubstituted.

When substituted, the substituent group(s) is/are preferably one or two groups independently selected from the group consisting of hydroxy; halo; lower alkyl; unsubstituted lower alkoxy; aryl optionally substituted with one or more groups, preferably one or two groups, which are independently of each other hydroxy, halo, unsubstituted lower alkyl or unsubstituted lower alkoxy groups; 6-membered heteroaryl having from 1 to 3 nitrogen atoms in the ring, the carbon atoms in the ring being optionally substituted with one or more groups, preferably one or two groups, which are independently of each other hydroxy, halo, unsubstituted lower alkyl or unsubstituted lower alkoxy groups; 5-membered heteroaryl having from 1 to 3 heteroatoms in the ring, selected from the groups consisting of nitrogen, oxygen and sulfur, the carbon and the nitrogen (if present) atoms in the ring being optionally substituted with one or more groups, preferably one or two groups, which are independently of each other hydroxy, halo, unsubstituted lower alkyl or unsubstituted lower alkoxy groups; a 5- or 6-membered heteroalicyclic group having from 1 to 3 heteroatoms in the ring, selected from the group consisting of nitrogen, oxygen and sulfur, the carbon and the nitrogen (if present) atoms in the ring being optionally substituted with one or two groups, preferably one or two groups, which are independently of each other hydroxy, halo, unsubstituted lower alkyl or unsubstituted lower alkoxy groups; mercapto; (unsubstituted lower alkyl)thio; arylthio optionally substituted with one or more groups, preferably one or two groups, which are independently of each other hydroxy, halo, unsubstituted lower alkyl or unsubstituted lower alkoxy groups; cyano; acyl; thioacyl; O-carbamyl; N-carbamyl; O-thiocarbamyl; N-thiocarbamyl; C-amido; N-amido; nitro; N-sulfonamido; S-sulfonamido; —S(O)R¹⁸; —S(O)₂R¹⁸; —C(O)OR¹⁸; —OC(O)R¹⁸ and —NR¹⁸R¹⁹; wherein R¹⁸ and R¹⁹ are as defined above.

“Alkenyl” refers to a lower alkyl group, as defined herein, consisting of at least two carbon atoms and at least one carbon-carbon double bond. Representative examples include, but are not limited to, ethenyl, 1-propenyl, 2-propenyl, 1-, 2- or 3-butenyl, and the like.

“Alkynyl” refers to a lower alkyl group, as defined herein, consisting of at least two carbon atoms and at least one carbon-carbon triple bond. Representative examples include, but are not limited to, ethynyl, 1-propynyl, 2-propynyl, 1-, 2- or 3-butynyl, and the like.

“Aryl” refers to an all-carbon monocyclic or fused polycyclic ring (a “fused” ring system means that each ring in the system shares an adjacent pair of atoms with another ring in the system) of 5-12 carbon atoms having a completely conjugated pi-electron system. Examples, without limitation, of aryl groups are phenyl, naphthalenyl and anthracenyl. The aryl groups may be substituted or unsubstituted. When substituted, the substituent group(s) is/are preferably one or more groups, more preferably one, two or three groups, even more preferably one or two groups, independently of each other selected from trihalomethyl; hydroxy; halo; unsubstituted lower alkyl; unsubstituted lower alkoxy; mercapto; (unsubstituted lower alkyl)thio; arylthio optionally substituted with one or more groups, preferably one or two groups, which are independently of each other selected from hydroxy, halo, unsubstituted lower alkyl or unsubstituted lower alkoxy groups; cyano; acyl; thioacyl; O-carbamyl; N-carbamyl; O-thiocarbamyl; N-thiocarbamyl; C-amido; N-amido; nitro; N-sulfonamido; S-sulfonamido; —S(O)R¹⁸; —S(O)₂R¹⁸; —C(O)OR¹⁸; —OC(O)R¹⁸ and —NR¹⁸R¹⁹; wherein R¹⁸ and R¹⁹ are as defined above. Preferably the aryl group is optionally substituted with one or two substituents independently selected from hydroxy, halo, unsubstituted lower alkyl, unsubstituted lower alkoxy, cyano, mercapto, N-amido, mono- or dialkylamino, carboxyl or N-sulfonamido.

“Heteroaryl” refers to a monocyclic or fused polycyclic ring (a “fused” ring system means that each ring in the system shares an adjacent pair of atoms with another ring in the system) of 5-12 ring atoms containing one, two or three ring heteroatoms selected from N, O or S, the remaining ring atoms being C, and in addition having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, iso-quinoline, purine and carbazole. The heteroaryl group may be substituted or unsubstituted. When substituted, the substituent group(s) is/are preferably one or more groups, more preferably one, two or three groups, even more preferably one or two groups, independently of each other selected from trihalomethyl, hydroxy, halo, unsubstituted lower alkyl, unsubstituted lower alkoxy, mercapto, (unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, —S(O)R¹⁸, —S(O)₂R¹⁸, —C(O)OR¹⁸, —OC(O)R¹⁸ and —NR¹⁸R¹⁹, wherein R¹⁸ and R¹⁹ are as defined above. Preferably the heteroaryl group is optionally substituted with one or two substituents independently selected from hydroxy, halo, unsubstituted lower alkyl, trihalomethyl, cyano, mercapto, N-amido, mono- or dialkylamino, carboxyl or N-sulfonamido.

“Heteroalicyclic” refers to a monocyclic or fused polycyclic ring group having 5-9 ring atoms of which one or two are ring heteroatoms selected from N, O, or S(O)ₙ, where n is an integer from 0 to 2, the remaining ring atoms being C. The ring(s) may also have one or more double bonds. However the ring(s) does/do not have a completely conjugated pi-electron system. Examples, without limitations, of unsubstituted heteroalicyclic groups are pyrrolidino, piperidino, piperazino, morpholino, thiomorpholino, homopiperazino, and the like. The heteroalicyclic ring may be substituted or unsubstituted. When substituted, the substituent group(s) is/are preferably one or more groups, more preferably one, two or three groups, even more preferably one or two groups, independently of each other selected from trihalomethyl, hydroxy, halo, unsubstituted lower alkyl, unsubstituted lower alkoxy, mercapto, (unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, —S(O)R¹⁸, —S(O)₂R¹⁸, —C(O)OR¹⁸, —OC(O)R¹⁸ and NR¹⁸R¹⁹, wherein R¹⁸ and R¹⁹ are as defined above. Preferably, the heteroalicyclic group is optionally substituted with one or two substituents independently selected from hydroxy, halo, unsubstituted lower alkyl, trihalomethyl, cyano, mercapto, N-amido, mono- or dialkylamino, carboxyl or N-sulfonamido.

“Heterocycle” means a saturated cyclic radical of 3-8 ring atoms of which one or two are ring heteroatoms selected from N, O or S(O)ₙ, where n is an integer from 0 to 2, the remaining ring atoms being C, where 1 or 2 C atoms may optionally be replaced by a carbonyl group. The heterocycle ring may optionally be substituted with one, two or three substituents independently selected from optionally substituted lower alkyl (optionally substituted with one or two substituents independently selected from carboxyl or ester), haloalkyl, cyanoalkyl, halo, nitro, cyano, hydroxy, alkoxy, amino, monoalkylamino, dialkylamino, aralkyl, heteroalkyl, heteroaralkyl, —COR (where R is a alkyl) or —COOR (where R is hydrogen or alkyl). More specifically the term heterocycle includes, but is not limited to, tetrahydropyranyl, 2,2-dimethyl-1,3-dioxolanyl, piperidino, N-methyl-piperidin-3-yl, piperazino, pyrrolidino, morpholino, thiomorpholino, thiomorpholino-1-oxide, thiomorpholino-1,1-dioxide, 4-ethyloxycarbonyl-piperazino, 3-oxo-piperazino, 2-imidazolinonyl, 2-pyrrolidinonyl, 2-oxo-homopiperazino, tetrahydropyrimin-2-onyl, and derivatives thereof. Preferably, the heterocycle group is optionally substituted with one or two substituents independently selected from halo, unsubstituted lower alkyl and lower alkyl substituted with carboxyl, ester, hydroxy, mono- or dialkylamino.

“Carboxyl” means a —COOH group.

“Hydroxy” means —OH group.

“Alkoxy” preferably refers to both an —O-(unsubstituted alkyl) and an —O-(unsubstituted cycloalkyl) group, but may also refer to both an O-(substituted alkyl) and an —O-(substituted cycloalkyl) group. Representative examples include, but are not limited to, methoxy, ethoxy, propoxy, butoxy, cyclopropyloxy, cyclobutyloxy, cyclopentyloxy, cyclohexyloxy, and the like. An “alkoxide” is similarly defined as an alkoxy group with a negative charge on the oxygen.

“Aryloxy” refers to both an —O-aryl and —O-heteroaryl group, as defined herein. Representative examples include, but are not limited to, phenoxy, pyridinyloxy, furanyloxy, thienyloxy, pyrimidinyloxy, pyrazinyloxy, and the like, and derivatives thereof. An “aryloxide” is similarly defined as an aryloxy group with a negative charge on the oxygen.

“Mercapto” means —SH group.

“Alkylthio” preferably refers to both an —S-(unsubstituted alkyl) and an —S-(unsubstituted cycloalkyl) group, but may also refer to an —S-(substituted alkyl) and an —S-(substituted cycloalkyl) group. Representative examples include, but are not limited to, methylthio, ethylthio, propylthio, butylthio, cyclopropylthio, cyclobutylthio, cyclopentylthio, cyclohexylthio, and the like.

“Arylthio” preferably refers to both an —S-(unsubstituted aryl) and an —S-(unsubstituted aralkyl) group, but may also refer to both an —S-(substituted aryl) and an —S-(substituted aralkyl) group. Representative examples include, but are not limited to, phenylthio, pyridinylthio, furanylthio, thienylthio, pyrimidinylthio, and the like.

“Acyl” refers to a —C(O)R″ group, where R″ is selected from the group consisting of hydrogen; unsubstituted lower alkyl; trihalomethyl; unsubstituted cycloalkyl; aryl optionally substituted with one or more groups, more preferably one, two or three groups, selected from the group consisting of unsubstituted lower alkyl, trihalomethyl, unsubstituted alkoxy, halo and —NR¹⁸R¹⁹ groups; and heteroalicyclic (bonded through a ring carbon) optionally substituted with one or more groups, more preferably one, two or three groups, selected from the group consisting of unsubstituted lower alkyl, trihalomethyl, unsubstituted alkoxy, halo and —NR¹⁸R¹⁹ groups; wherein R¹⁸ and R¹⁹ are as defined above. Representative acyl groups include, but are not limited to, acetyl, trifluoroacetyl, benzoyl, and the like.

“Aldehyde” means an acyl group, wherein R″ is hydrogen.

“Thioacyl” refers to a —C(S)R″ group, wherein R″ is as defined above.

“Ester” means a —C(O)OR″ group, wherein R″ is as defined above except that R″ cannot be hydrogen.

“Acetyl” refers to a —C(O)CH₃ group.

“Halo” refers to fluorine, chlorine, bromine or iodine, preferably fluorine or chlorine.

“Trihalomethyl” refers to a —CX₃ group, wherein X is a halo group as defined above.

“Trihalomethylsulfonyl” refers to a —S(O)₂CX₃ groups, wherein X is a halo group as defined above.

“Cyano” refers to a —C≡N group.

“Methylenedioxy” refers to a —OCH₂O— group, where the two oxygen atoms are bonded to adjacent carbon atoms.

“Ethylenedioxy” refers to a —OCH₂CH₂O— group, where the two oxygen atoms are bonded to adjacent carbon atoms.

“S-sulfonamido” refers to a —S(O)₂NR¹⁸R¹⁹ group, wherein R¹⁸ and R¹⁹ are as defined above.

“N-sulfonamido” refers to a —NR¹⁸S(O)₂R¹⁹ group, wherein R¹⁸ and R¹⁹ are as defined above.

“O-carbamyl” refers to a —OC(O)NR¹⁸R¹⁹ group, wherein R¹⁸ and R¹⁹ are as defined above.

“N-carbamyl” refers to a —NR¹⁸C(O)OR¹⁹ group, wherein R¹⁸ and R¹⁹ are as defined above.

“O-thiocarbamyl” refers to a —OC(S)NR¹⁸R¹⁹ group, wherein R¹⁸ and R¹⁹ are as defined above.

“N-thiocarbamyl” refers to a —NR¹⁸C(S)OR¹⁹ group, wherein R¹⁸ and R¹⁹ are as defined above.

“Amino” refers to a —NR¹⁸R¹⁹ group, wherein R¹⁸ and R¹⁹ are as defined above.

“C-amido” refers to a —C(O)NR¹⁸R¹⁹ group, wherein R¹⁸ and R¹⁹ are as defined above.

“N-amido” refers to a —NR¹⁸C(O)R¹⁹ group, wherein R¹⁸ and R¹⁹ are as defined above.

“Nitro” refers to a —NO₂ group,

“Haloalkyl” means an otherwise unsubstituted alkyl, preferably an otherwise unsubstituted lower alkyl, which is substituted with one or more same or different halo atoms, e.g. —CH₂Cl, —CF₃, —CH₂CF₃, —CH₂CCl₃, and the like.

“Aralkyl” means an otherwise unsubstituted alkyl, preferably an otherwise unsubstituted lower alkyl, which is substituted with an aryl group, wherein said aryl group may be unsubstituted or further substituted, e.g. —(CH₂)-phenyl, —(CH₂)₂-phenyl, —(CH₂)₃-phenyl, —CH₂CH(CH₃)CH₂-phenyl, and the like.

“Heteroaralkyl” means an otherwise unsubstituted alkyl, preferably an otherwise unsubstituted lower alkyl, which is substituted with a heteroaryl group, wherein said heteroaryl group may be unsubstituted or further substituted, e.g. —(CH₂)-pyridinyl, —(CH₂)₂-pyrimidinyl, —(CH₂)₃-imidazolyl, and the like.

“Monoalkylamino” means a radical —NHR³⁰ where R³⁰ is an unsubstituted alkyl or unsubstituted cycloalkyl group as defined above, e.g. methylamino, (1-methylethyl)amino, cyclohexylamino, and the like.

“Dialkylamino” means a radical —N(R³⁰)₂ where each R³⁰ is independently an unsubstituted alkyl or unsubstituted cycloalkyl group as defined above, e.g. dimethylamino, diethylamino, (1-methylethyl)ethylamino, cyclohexylmethylamino, cyclopentylmethylamino, and the like.

“Cyanoalkyl” means an otherwise unsubstituted alkyl, preferably an otherwise unsubstituted lower alkyl, which is substituted with 1 or 2 cyano groups.

“Optional” or “optionally” means that the subsequently described event or circumstance may but need not occur and that the description includes instances in which it does not For example, “heterocycle optionally substituted with an alkyl group” means that the alkyl group may but need not be present and that the description includes situations where the heterocycle group is not substituted with the alkyl group.

The terms “2-indolinone”, “indolin-2-one” and “2-oxindole” are used interchangeably herein to refer to a compound having the chemical structure:

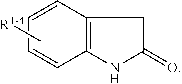

The term “pyrrole” refers to a compound having the chemical structure:

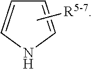

The terms “pyrrole substituted 2-indolinone” and “3-pyrrolidenyl-2-indolinone” are used interchangeably herein to refer to a compound having the chemical structure shown in formula (I):

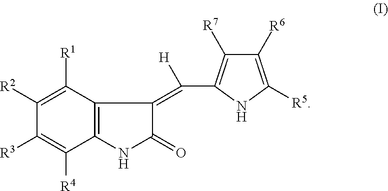

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, an atom bonded to four different groups, the compound may have a pair of enantiomers. An enantiomer can be characterized by the absolute configuration of its asymmetric center, and is described by the R- and S-sequencing rules of Cahn and Prelog, or by the manner in which the compound rotates plane-polarized light and designated as dextrorotatory or levorotatory (i.e. as (+) or (−)-isomers respectively). A chiral compound can exist as either individual enantiomer or as a mixture, for example, a mixture containing equal proportions of the enantiomers called a “racemic mixture”.

The compounds of this invention may possess one or more asymmetric centers; such compounds can therefore be produced as individual (R)- or (S)-stereoisomers or as mixtures thereof. For example, if the R⁶ substituent in a compound of formula (I) is 1-hydroxyethyl, then the carbon to which the hydroxy group is attached is an asymmetric center and therefore the compound of formula (I) can exist as an (R)- or (S)-stereoisomer.

Unless indicated otherwise, the description or naming of a particular compound in the specification and claims is intended to include both individual enantiomers and mixtures thereof, racemic or otherwise.

The compounds of formula (I), including (Ia), (Ib), etc. may exhibit the phenomenon of tautomerism and structural isomerism. For example, the structures described herein may adopt an E or a Z configuration about the double bond connecting the 2-indolinone moiety to the pyrrole moiety or they may be a mixture of E and Z. This invention encompasses any tautomeric or structural isomeric form and mixtures thereof which possess the ability to modulate RTK, CTK and/or STK activity and is not limited to any one tautomeric or structural isomeric form.

A “pharmaceutical composition” refers to a mixture of one or more of the compounds described herein, or physiologically or pharmaceutically acceptable salts or prodrugs or metabolites thereof, with other chemical components such as physiologically or pharmaceutically acceptable carriers and excipients. The purpose of a pharmaceutical composition is to facilitate administration of a compound to an organism.

The compounds of formula (I) may also act as a prodrug. A “prodrug” refers to an agent, which is converted into the parent drug in vivo. Prodrugs are often useful because in some situations, they may be easier to administer than the parent drug. They may for instance be bioavailable by oral administration whereas the parent drug is not. A prodrug may also have improved solubility in pharmaceutical compositions compared to the parent drug.

Additionally, it is contemplated that a compound of formula (I) would be metabolized by enzymes in the body of an organism such as a human being to generate a metabolite that can modulate the activity of the protein kinases. Such metabolites are within the scope of the present invention.

A physiologically or pharmaceutically acceptable carrier refers to a carrier or diluent that does not cause significant irritation to an organism and does not abrogate the biological activity and properties of the administered compound.

A pharmaceutically acceptable excipient refers to a preferably inert substance that is added to a pharmaceutical composition to further facilitate administration of a compound. Examples without limitation of excipients include calcium carbonate, calcium phosphate, various sugars and types of starch, cellulose derivatives, gelatin, vegetable oils and polyethylene glycols.

As used herein, the term “pharmaceutically acceptable sale” refers to those salts, which retain the biological effectiveness and properties of the parent compound. Such salts are preferably non-toxic.

Salts according to the invention include:

(i) acid addition salts which are obtained by the reaction of the free base of the parent compound with inorganic acids such as hydrochloric acid, hydrobromic acid, nitric acid, phosphoric acid, sulfuric acid, perchloric acid and the alike, or with organic acids such as acetic acid, oxalic acid, (D)- or (L)-malic acid, maleic acid, methanesulfonic acid, salicylic acid, tartaric acid, citric acid, succinic acid, malonic acid and alike, preferably hydrochloric acid or (L)-malic acid, more preferably (L)-malic acid (such as to provide the L-malate salt of 5-(5-fluoro-2-oxo-1,2-dihydroindol-3-ylidenemethyl)-2,4-dimethyl-1H-pyrrole-3-carboxylic acid (2-diethylamioethyl)amide); or

(ii) salts formed when an acidic proton present in the parent compound either is replaced by a metal ion (e.g. an alkaline metal ion, an alkaline earth metal ion or an aluminium ion) or coordinates with an organic base (such as ethanolamine, diethanolamine, triethanolamine, tromethamine, N-methylglucamine, and the like).

Scheme I illustrates a general reaction scheme for carrying out a preferred method of the invention.

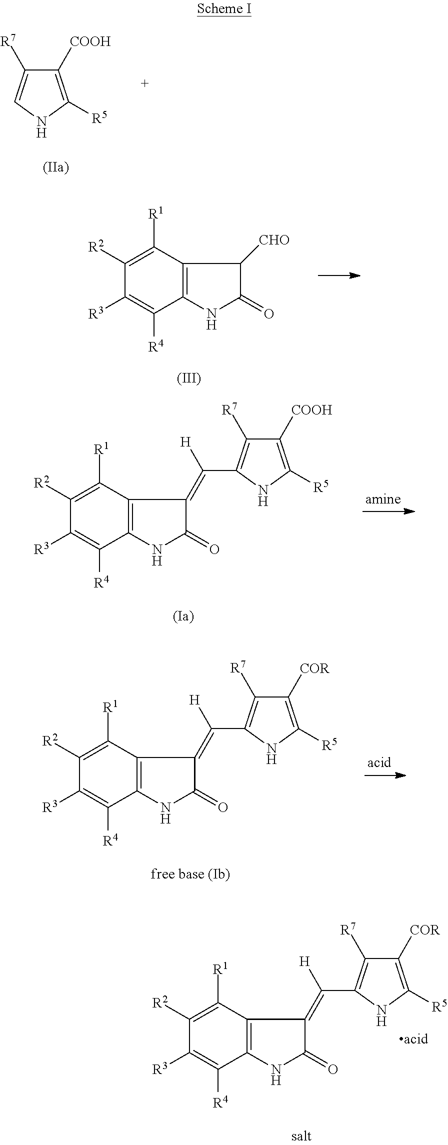

The acid (IIa) may be reacted with the aldehyde (III) in the presence of an acidified polar solvent system to form the pyrrole substituted 2-indolinone (Ia). In preferred embodiments, the polar solvent system comprises one or more hydroxylic solvent(s). Preferably the solvent is a hydroxylic organic solvent, most preferably ethanol. In another embodiment, the acid is selected from the group comprising mineral acids, for example, hydrochloric acid, concentrated hydrochloric acid, sulfuric acid, concentrated sulfuric acid, and organic acids such as glacial acetic acid, p-toluene sulfonic acid. Preferably the acid is hydrochloric acid, in particular when the solvent is ethanol, Non-limiting examples of acidified polar solvent systems according to the invention include:

1. ethanol containing a catalytic amount of glacial acetic acid;

2. ethanol containing a catalytic amount of concentrated hydrochloric acid;

3. ethanol containing a catalytic amount of concentrated sulfuric acid;

4. ethanol containing a catalytic amount or molar equivalent of p-toluene sulfonic acid;

5. tetrahydrofuran containing a catalytic amount of concentrated hydrochloric acid;

6. acidic tetrahydrofuran (i.e. HCl purged in THF);

7. tetrahydrofuran containing a catalytic amount of glacial acetic acid;

8. tetrahydrofuran containing a catalytic amount of concentrated sulfuric acid;

9. toluene containing a catalytic amount or molar equivalent of p-toluene sulfonic acid;

10. iso-propyl alcohol (IPA) containing a catalytic amount of glacial acetic acid;

11. IPA containing a catalytic amount of concentrated hydrochloric acid;

12. IPA containing a catalytic amount of concentrated sulfuric acid;

13. IPA containing a catalytic amount or molar equivalent of p-toluene sulfonic acid;

14. acidic IPA (i.e. HCl purged in IPA);

15. methanolic hydrogen chloride;

and similar solvent systems that are within the scope of the invention and are within the skill set of the notional skilled person without inventive capacity to determine. Most preferably the acidified solvent system is ethanolic hydrogen chloride.

In preferred embodiments, the reaction mass obtained from the coupling of intermediates (IIa) and (III) can be diluted, preferably with an aqueous base. Any type of base may be used, non-limiting examples include aqueous solutions of potassium bicarbonate, sodium carbonate, potassium carbonate, sodium hydroxide and potassium hydroxide. In certain embodiments, the resulting product (Ia) may be isolated, preferably by filtration and drying under reduced pressure.

The acid (Ia) may then be reacted with the desired amine to form the corresponding product with the desired amide substitution. For example, in a particularly preferred embodiment, when scheme I is employed to prepare sunitinib, the amine added is N,N-diethylethylenediamine. This reaction preferably takes place in a solvent which preferably is a polar aprotic solvent such as THF, DMF or an ether. A number of further reagents may be added during this reaction. In preferred embodiments, a coupling agent may be added to the reaction mixture, for example, N,N′-dicyclohexylcarbodiimide (DCC), 1-ethyl-3-(3-dimethylaminopropyl)carbodiimide hydrochloride (EDC.HCl) or N,N′-carbonyldiimidazole (CDI), preferably together with a suitable organic base such as a tertiary or aromatic amine. Suitable organic bases include 4-dimethylaminopyridine (DMAP), N-methyl-morpholine, trimethylamine, pyridine, 1,8-diazabicyclo[5.4.1]undec-7-ene, pyrolidone, N-methyl-piperidone, diisopropylethylamine and triethylamine (TEA). Preferably a catalyst such as 1-hydroxybenzotriazole (HOBT) is also used. In certain preferred embodiments, the reaction mass comprising the acid (Ia) and the coupling agent and optionally the base and catalyst may be refluxed for preferably between about 1-10 hours, most preferably between about 3-5 hours.

The desired product is then isolated by any suitable means. For example, the reaction mass may be extracted by any suitable solvent. The inventors have found that when sunitinib is prepared, extraction with ethyl acetate is particularly suitable. The extracted layer is then separated and dried. For example, in the continued example of sunitinib extracted in ethyl acetate the product may be dried over anhydrous sodium and/or magnesium sulfate, wherein subsequent filtration and evaporation of the ethyl acetate yields the desired product. Of course, the skilled person will understand that there are a number of techniques that may be employed to isolate the desired compound.

Scheme II illustrates an alternative method of the general reaction for carrying out the methods of the invention.

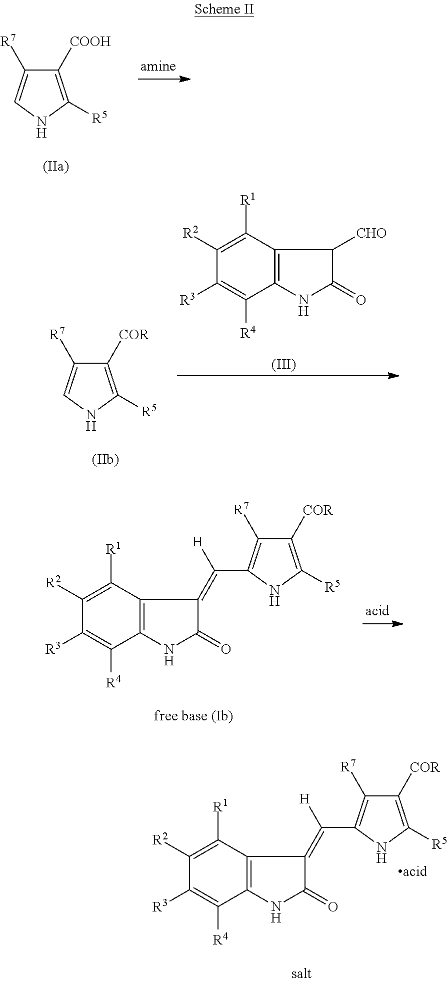

The acid (IIa) is reacted with the desired amine, for example, when the compound sunitinib is prepared, the desired amine is N,N-diethylethylenediamine, to form the amide (IIb). A similar reaction, albeit with a formyl-substituted pyrrole, is described in more detail and exemplified, for example, in WO 01/60814 which is incorporated herein by reference in its entirety.

The reaction in preferred embodiments may be carried out in a polar aprotic solvent which in further preferred embodiments may be selected from the group comprising THF, diethyl ether, methyl t-butyl ether, acetonitrile (ACN) and DMF. Further polar aprotic solvents may be employed within the scope of the invention. Preferably, the reaction is carried out at ambient temperatures, for example, between about 20-30° C., although the person skilled in the art will appreciate that the reaction may be conducted at different temperatures.

The amide (IIb) is then reacted with the aldehyde (III) to form the free base product (Ib). Preferably the reaction occurs in an acidified polar solvent system. Preferably the solvent is a hydroxylic organic solvent, most preferably ethanol. In another embodiment, the acid is selected from the group comprising mineral acids, for example, hydrochloric acid, concentrated hydrochloric acid, sulfuric acid, concentrated sulfuric acid, and organic acids such as glacial acetic acid, p-toluene sulfonic acid. Preferably the acid is hydrochloric acid, in particular when the solvent is ethanol. Non-limiting examples of acidified polar solvent systems according to the invention include:

1. ethanol containing a catalytic amount of glacial acetic acid;

2. ethanol containing a catalytic amount of concentrated hydrochloric acid;

3. ethanol containing a catalytic amount of concentrated sulfuric acid;

4. ethanol containing a catalytic amount or molar equivalent of p-toluene sulfonic acid;

5. tetrahydrofuran containing a catalytic amount of concentrated hydrochloric acid;

6. acidic tetrahydrofuran (i.e. HCl purged in THF);

7. tetrahydrofuran containing a catalytic amount of glacial acetic acid;

8. tetrahydrofuran containing a catalytic amount of concentrated sulfuric acid;

9. toluene containing a catalytic amount or molar equivalent of p-toluene sulfonic acid;

10. iso-propyl alcohol (IPA) containing a catalytic amount of glacial acetic acid;

11. IPA containing a catalytic amount of concentrated hydrochloric acid;

12. IPA containing a catalytic amount of concentrated sulfuric acid;

13. IPA containing a catalytic amount or molar equivalent of p-toluene sulfonic acid;

14. acidic IPA (i.e. HCl purged in IPA);

15. methanolic hydrogen chloride;

and similar solvent systems that are within the scope of the invention and are within the skill set of the notional skilled person without inventive capacity to determine. Most preferably the acidified solvent system is ethanolic hydrogen chloride.

In preferred embodiments, the reaction mass obtained can be diluted, preferably with a base. Any type of base may be used, non-limiting examples include potassium bicarbonate, sodium carbonate, potassium carbonate, sodium hydroxide and potassium hydroxide.

If desired, the free base product (Ib), however prepared, can in preferred embodiments be further reacted with a suitable acid to form a salt, preferably a pharmaceutically acceptable salt. In a preferred embodiment, the salt prepared is the malic acid salt, by reaction with malic acid. Particularly preferred is preparation of the L-malic acid salt. Although in certain other embodiments non-pharmaceutically acceptable salts maybe be prepared as intermediates in the preparation of pharmaceutically acceptable compounds.

The solid obtained from the above described procedures may be isolated by any suitable means. The inventors have found that filtering under conditions of reduced pressure, preferably under vacuum is particularly advantageous. The filtered solid may then be washed and dried.

For the purposes of the present invention, a compound is “substantially pure”, if it comprises less than 1% impurity by HPLC, preferably less than 0.5%, preferably less than 0.3%, preferably less than 0.2%, preferably less than 0.1%. In preferred embodiments the compounds of the invention are substantially pure.

The present inventors have surprisingly found that the invention includes the advantages of large reductions in reaction time as compared to the prior art processes and results in a compound of very high purity (>99% by HPLC).

In the present invention, the novel synthetic intermediate products are not purified. However, as part of the present invention, the synthetic intermediates may be purified if so desired. Any suitable purification technique may be employed, for example, recrystallisation from suitable solvents.

The pharmaceutical composition of the present invention can be a solution or suspension, but is preferably a solid dosage form such as a solid oral dosage form. Preferred oral dosage forms in accordance with the invention include tablets, capsules and the like which, optionally, may be coated if desired. Tablets can be prepared by conventional techniques, including direct compression, wet granulation and dry granulation. Capsules are generally formed from a gelatin material and can include a conventionally prepared granulate of excipients in accordance with the invention.

The pharmaceutical composition according to the present invention typically comprises one or more conventional pharmaceutically acceptable excipient(s) selected from the group comprising a filler, a binder, a disintegrant, a lubricant, and optionally further comprises at least one excipient selected from colouring agents, adsorbents, surfactants, film-formers and plasticizers.

If the solid pharmaceutical formulation is in the form of coated tablets, the coating may be prepared from at least one film-former such as hydroxypropyl methyl cellulose, hydroxypropyl cellulose or methacrylate polymers which optionally may contain at least one plasticizer such as polyethylene glycols, dibutyl sebacate, triethyl citrate, and other pharmaceutical auxiliary substances conventional for film coatings, such as pigments, fillers and others.

It is also envisaged that in certain embodiments the compositions according to the invention may comprise a second or further active ingredient(s).

The details of the invention, its objects and advantages are illustrated below in greater detail by the following non-limiting examples.

### EXAMPLES

### Example 1

### Preparation of Pyrrole Intermediate

### Example 1a

### 2,4-dimethyl-1H-pyrrole-3-carboxylic acid

To a solution of ethyl 2,4-dimethyl-1H-pyrrole-3-carboxylate (1 eq) in methanol (3 vol) was added a solution of KOH (2-3 eq) in water (0.4 vol) and the reaction mass was refluxed for approximately 5-6 hours. After the reaction was complete as indicated by thin layer chromatography (TLC), the heating was stopped and the reaction mass cooled to ambient temperature. The reaction mass was washed with ethyl acetate (2×3 vol), and the aqueous layer was collected and acidified with 1:1 conc. HCl:water v/v (3 vol) to pH 3-4. The resultant solid precipitate was filtered with a Buchner funnel under vacuum and then dried on a rotavapor under reduced pressure at 40° C. for 5-6 hours.

Molar yield=76%.

HPLC purity=96.45%.

IR (KBr) cm⁻¹: 3366 (broad, O—H), 2951, 2922, 2679, 2638, 1654 (C═O), 1647, 1578, 1524, 1508, 1481, 1466, 1449, etc.

¹H-NMR (DMSO-d₆) δ ppm: 2.05 (s, 3H, —CH₃), 2.10 (s, 3H, —CH), 6.36 (s, 1H, ═C—H), 10.79 (s, 1H, NH, D₂O exchangeable), 11.40 (s, 1H, —OH, D₂O exchangeable).

¹³C-NMR (DMSO-d₆) δ ppm: 12.70 (1C, —CH₃, DEPT), 13.60 (1C, —CH₃, DEPT), 110.09 & 119.82 (2C, 2×—C—CH₃), 114.59 (1C, —CH—, DEPT), 135.25 (1C, —C—CO—), 167.11 (1C, —CO—OH).

Mass (m/z): (M+1) 140 (100%).

### Example 1b

### N-(2-(diethylamino)ethyl)-2,4-dimethyl-1H-pyrrole-3-carboxamide

2,4-Dimethyl-1H-pyrrole-3-carboxylic acid (1 eq) was added to a solution of THF (15 vol), 1-ethyl-3-(3-dimethylaminopropyl)carbodiimide hydrochloride (EDC.HCl) (1.5 eq), 1-hydroxybenzotriazole (HOBT) 1.5 eq) and TEA (2 eq) at ambient temperature and stirred for 15-30 minutes. To this solution was added N,N-diethylethylenediamine (3 eq) and the reaction mass was stirred for 8-10 hours. After the reaction was complete as indicated by TLC, THF was distilled out at reduced pressure and the reaction mass was then diluted by adding a saturated sodium bicarbonate solution (any inorganic weak base such as potassium carbonate, potassium bicarbonate, sodium carbonate, etc or even dilute NaOH or KOH solution may be used) (3 vol) and the pH adjusted to 7-10. The whole mass was extracted with ethyl acetate (2×5 vol), which was separated, dried over anhydrous sodium sulfate then filtered. The ethyl acetate was distilled out to obtain a brown viscous mass.

Molar yield=93%.

HPLC purity=93.42%.

IR (KBr) cm⁻¹: 3241 (broad, N—H), 3063, 2967, 2927, 2872, 2818, 1622 (C═O), 1575, 1530, 1504, 1455, 1401, etc.

¹H-NMR (DMSO-d₆) δ ppm: 0.96 (t, J=7.1 Hz, 6H, 2×—CH₂—CH₃), 2.08 (s, 3H, —CH₃), 2.29 (s, 3H, —CH₃), 2.46-2.52 (m, 6H, 3×—N—CH₂), 3.21-3.27 (m, 2H, —CO—NH—CH₂—), 6.33 (s, 1H, ═C—H), 6.78 (s, 1H, amide NH, D₂O exchangeable), 10.55 (s, 1H, pyrrole NH, D₂O exchangeable).

¹³C-NMR (DMSO-d₆) δ ppm: 11.90 (2C, 2×—CH₂—CH₃, DEPT), 12.04 (1C, —CH₃, DEPT), 12.81 (1C, —CH₃, DEPT), 36.59 (1C, —CH₂—, DEPT), 46.40 (2C, 2×—CH₂—, DEPT), 51.67 (1C, —CH₂—, DEPT), 114.14 (1C, —CH—, DEPT), 115.01 & 116.33 (2C, 2×—C—CH₃), 130.04 (1C, —C—CO—), 165.85 (1C, C═O),

Mass (m/z): (M−1) 238 (100%).

### Example 2

### Preparation of 2-oxindole with aldehyde substitution at 3-position (5-fluoro-3-formyl-1H-indole-2-one)

### Example 2a

### 2-oxindole from 5-fluoro-isatin

Hydrazine hydrate (7 eq, 103.2 ml) was charged to a four-neck round-bottomed flask equipped with a reflux condenser, mechanical stirrer and oil bath. To this was added 5-fluoro-isatin (0.4 eq, 20 g) and the mixture was stirred and heated to 100° C. After the desired temperature was reached, more 5-fluoro-isatin was added in four portions (0.15 eq, 7.5 g at each portion). In total 1 eq=50 g of 5-fluoro-isatin was added. After the complete addition of the 5-fluoro-isatin, the reaction mass was maintained at the same temperature for 3 hours and then allowed to cool to ambient temperature (25-30° C.). Acidified water (concentrated HCl 5 vol+water 3.3 vol) was added and stirring continued for about 24 hours. The solid obtained was filtered using a Buchner funnel under vacuum and washed with water twice (2×7.5 vol) and then dried in a vacuum oven at 0.5 kg/cm² at 55° C. for 5 hours.

Molar yield=75%.

HPLC=98.85%.

IR (KBr) cm⁻¹: 3215, 3079, 3053, 2931, 2881, 1699, 1669, 1631, 1484, etc.

¹H-NMR (DMSO-d₆) δ ppm: 3.50 (s, 2H, —CH₂—), 6.76-6.80 (dd, J=4.5 Hz, 1H, Ar—H), 6.99 (m, 1H, Ar—H), 7.12 (dd, J=2.5 Hz, 1H, Ar—H), 10.38 (s, 1H, NH, D₂O exchangeable).

¹³C-NMR (DMSO-d₆) δ ppm: 36.20 (1C, —CH₂—), 109.52-159.29 (6C, 6×Ar—C), 176.23 (1C, C═O).

Mass (m/z): (M+1) 152 (100%).

### Example 2b

### 5-fluoro-3-formyl-1H-indole-2-one (or 5-fluoro-3-formyl-2-oxindole) from 2-oxindole

Methanol (5-vol) was charged to a four-neck 250 ml round-bottomed flask equipped with a mechanical stirrer, water bath and reflux condenser. Sodium methoxide (2.1 eq) was added to it and the mixture stirred to obtain a clear solution. To the clear solution, 2-oxindole (1 eq, 15 g) and ethyl formate (2.9 eq, 23.27 ml) were added and then stirring was continued at reflux temperature for about 1 hour. The mixture was allowed to cool to ambient temperature (25-30° C.). The reaction mass was poured in ice-cold water (2 vol, 100 ml) under stirring and the pH adjusted to pH 3 by the addition of 1:1 conc. HCl: water v/v (approximately 35 ml). The stirring was continued for 30 minutes and the resultant solid filtered on a Buchner funnel under vacuum and dried in a vacuum oven at 0.5 kg/cm² at 55° C. for 5 hours.

Molar yield=98%.

HPLC=98.90%.

IR (KBr) cm⁻¹: 3190, 3020, 2721, 1692, 1624, 1601, 1565, 1467, etc.

¹H-NMR (DMSO-d₆) δ ppm: 6.75-6.79 (dd, J=4.6 Hz, 1H, Ar—H), 6.85-6.92 (m, 1H, Ar—H), 7.24-7.27 (dd, J=2.4 Hz, 1H, Ar—H), 7.86 (bs, 1H, —CHO), 10.20 (s, 1H, NH, D₂O exchangeable).

¹³C-NMR (DMSO-d₆) δ ppm: 106.04 (1C, —CH—CHO), 108.39-156.42 (6C, 6×Ar—C), 159.18 (1C, —CHO) 169.90 (1C, C═O).

Mass (m/z): (M+1) 180 (100%).

### Example 3

### Preparation of Example Compound of the Invention (Sunitinib)

### Example 3a

### N-[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib)

N-(2-(Diethylamino)ethyl)-2,4-dimethyl-1H-pyrrole-3-carboxamide (1 eq) and 5-fluoro-3-formyl-2-oxindole (1 eq) were refluxed together in ethanolic hydrogen chloride (5% w/w, 15 vol) for 6-12 hours. After completion of the reaction as indicated by TLC, the reaction mass was diluted with saturated sodium bicarbonate solution (10 vol) and the pH adjusted to pH 9-10. The solid thus obtained was filtered on a Buchner funnel under vacuum and washed with ethanol (5 vol) and dried in a vacuum oven at 0.5 kg/cm² at 55° C. for 5 hours to afford a yellow-orange solid.

Molar yield=75%.

HPLC purity=93.87%.

IR (KBr) cm⁻¹: 3276 (broad, N—H), 3063, 2966, 2925, 2807, 1675 (C═O), 1560, 1475, etc.

¹H-NMR (DMSO-d₆) δ ppm: 0.97 (t, J=7.1 Hz, 6H, 2×—CH₂—CH₃), 2.42 (s, 3H, —CH₃), 2.44 (s, 3H, —CH₃), 2.47-2.56 (m, 6H, 3×—N—CH₂—), 3.25-3.31 (m, 2H, —CO—NH—CH₂), 6.83-6.87 (m, 1H, vinyl proton), 6.90-6.94 (t, J=5.9 Hz, 1H, aromatic ortho position), 7.43-7.47 (t, J=5.6 Hz, 1H, aromatic meta position), 7.74-7.78 (dd, J=5.9 Hz, 1H, aromatic ortho position), 7.72 (s, 1H, amide NH, D₂O exchangeable), 10.90 (s, 1H, pyrrole NH, D₂O exchangeable), 13.68 (s, 1H, indole NH, D₂O exchangeable).

¹³C-NMR (DMSO-d₆) δ ppm: 10.64 (1C, —CH₃, DEPT), 11.92 (2C, 2×—CH₂—CH₃, DEPT), 13.38 (1C, —CH₃, DEPT), 37.02 (1C, —CH₂—, DEPT), 46.55 (2C, 2×—CH₂—, DEPT), 51.69 (1C, —CH₂—, DEPT), 105.90 (1C, d, phenyl carbon, DEPT), 110.10 (1C, d, phenyl carbon, DEPT), 112.45 (1C, d, phenyl carbon, DEPT), 124.94 (1C, vinyl carbon, DEPT), 158.3 (1C, d, C—F, DEPT), 114.60 (bridge-head C of indole ring adjacent to >NH), 120.80, 134.50, 125.80, 136.70 (4C, pyrrole ring), 164.60 (1C, C═O), 169.63 (1C, C═O).

Mass (m/z): (M+1) 399 (100%), [(M+2)+1] 401 (14%).

### Example 3b

### 5-[(Z)-(5-fluoro-2-oxo-1,2-dihydro-3H-indol-3-ylidene)methyl]-2,4-dimethyl-1H-pyrrole-3-carboxylic acid

2,4-Dimethyl-1H-pyrrole-3-carboxylic acid (1 eq) and 5-fluoro-3-formyl-2-oxindole (1 eq) were refluxed together in ethanolic hydrogen chloride (5% w/w, 5 vol) for 6 hours. After completion of the reaction as indicated by TLC, the reaction mass was diluted with saturated sodium bicarbonate solution (10 vol) and the pH adjusted to pH 9-10. The solid obtained was filtered and dried under reduced pressure on a rotavapor at 40° C. to afford a yellow-orange solid.

Molar yield=65%.

HPLC purity=94.24%.

IR (KBr) cm⁻¹: 3437 (broad, N—H, O—H), 3160, 3101, 3041, 2953, 2922, 2873, 1668 (C═O), 1619, 1556, 1474, etc.

¹H-NMR (DMSO-d₆) δ ppm: 2.33 (s, 3H, —CH₃), 2.38 (s, 3H, —CH₃), 6.00 (s, 1H, vinyl proton), 6.79-6.82 (m, 2H, aromatic ortho+meta position), 7.15-7.19 (dd, J=5.0 Hz, 1H, aromatic ortho position), 7.33 (s, 1H, NH, D₂O exchangeable), 7.73 (s, 1H, NH, D₂O exchangeable), 13.14 (s, 1H, —OH, D₂O exchangeable),

¹³C-NMR (DMSO-d₆) δ ppm: 11.32 (1C, —CH₃), 13.57 (1C, —CH₃), 105.33 (1C, d, Ar—C, ortho position), 109.76 (1C, d, Ar—C, meta position), 111.62 (1C, d, Ar—C, ortho position), 112.90 (1C, vinyl carbon), 127.54 (1C, C—F), 111.62-159.71 (7C, 6×Ar—C, —CO—C═C), 159.71 (1C, C═O), 169.58 (1C, C═O).

Mass (m/z): (M+1) 299 (100%), [(M+1)+2] (13%).

### Example 3c

### N-[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib)

To a stirred solution of 5-[(Z)-(5-fluoro-2-oxo-1,2-dihydro-3H-indol-3-ylidene)methyl]-2,4-dimethyl-1H-pyrrole-3-carboxylic acid (1 eq) in THF (15 vol) was added 1-ethyl-3-(3-dimethylaminopropyl)carbodiimide hydrochloride (EDC.HCl) (1.5 eq), 1-hydroxybenzotriazole (HOBT) (1.5 eq) and TEA (2 eq) and the solution was stirred at room temperature for 30 minutes. To this solution was added N,N-diethylethylenediamine (2 eq) and the whole mass was stirred at room temperature for 8-10 hours. The reaction mass was then diluted with saturated sodium bicarbonate (8-10 vol) and the pH adjusted to pH 10 with the addition of a 50% NaOH aqueous solution (8-10 vol). The whole mass was then extracted with ethyl acetate (3×5 vol). The ethyl acetate layer was separated, dried over anhydrous sodium sulfate, then filtered. Evaporation of the ethyl acetate afforded the corresponding product.

Molar yield=70%.

HPLC purity=95.63%.

IR (KBr) cm⁻¹: 3276 (broad, N—H), 3063, 2966, 2925, 2807, 1675 (C═O), 1560, 1475, etc.

¹H-NMR (DMSO-d₆) δ ppm: 0.97 (t, J=7.1 Hz, 6H, 2×—CH₂—CH₃), 2.42 (s, 3H, —CH₃), 2.44 (s, 3H, —CH₃), 2.47-2.56 (m, 6H, 3×—N—CH₂—), 3.25-3.31 (m, 2H, —CO—NH—CH₂—), 6.83-6.87 (m, 1H, vinyl proton), 6.90-6.94 (t, J=5.9 Hz, 1H, aromatic ortho position), 7.43-7.47 (t, J=5.6 Hz, 1H, aromatic meta position), 7.74-7.78 (dd, J=5.9 Hz, 1H, aromatic ortho position), 7.72 (s, 1H, amide NH, D₂O exchangeable), 10.90 (s, 1H, pyrrole NH, D₂O exchangeable), 13.68 (s, 1H, indole NH, D₂O exchangeable).

¹³C-NMR (DMSO-d₆) δ ppm: 10.64 (1C, —CH₃, DEPT), 11.92 (2C, 2×—CH₂—CH₃, DEPT), 13.38 (1C, —CH₃, DEPT), 37.02 (1C, —CH₂—, DEPT), 46.55 (2C, 2×—CH₂—, DEPT), 51.69 (1C, —CH₂—, DEPT), 105.90 (1C, d, aromatic ortho position, DEPT), 110.10 (1C, d, aromatic meta position, DEPT), 112.45 (1C, d, aromatic meta position, DEPT), 124.94 (1C, vinyl carbon, DEPT), 158.3 (1C, d, C—F, DEPT), 114.60 (bridge-head C of indole ring adjacent to >NH), 120.80, 134.50, 125.80, 136.70 (4C, pyrrole ring), 164.60 (1C, C═O), 169.63 (1C, C═O).

Mass (m/z): (M+1) 399 (100%), [(M+2)+1] 401 (14%).

It will be readily apparent to one skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group.

1.-58. (canceled)

59. A process for the preparation of a 3-pyrrole substituted 2-indolinone of formula (I): 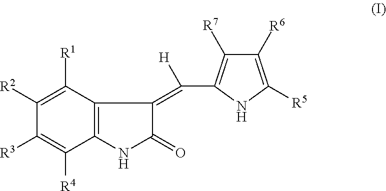
wherein:
R¹ is selected from the group consisting of hydrogen, halo, alkyl, cycloalkyl, aryl, heteroaryl, heteroalicyclic, hydroxy, alkoxy, —C(O)R¹⁵, —NR¹³R¹⁴, —(CH₂)ᵣR¹⁶ and —C(O)NR⁸R⁹;
R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, —S(O)₂NR¹³R¹⁴ and —SO₂R²⁰;
R³ is selected from the group consisting of hydrogen, halo, alkyl, trihalomethyl, hydroxy, alkoxy, —C(O)R¹⁵, —NR¹³R¹⁴, —NR¹³C(O)R¹⁴, —NR¹³C(O)OR¹⁴ and —SO₂R²⁰;
R⁴ is selected from the group consisting of hydrogen, halo, alkyl, hydroxy, alkoxy and —NR¹³R¹⁴;
R⁵ is selected from the group consisting of hydrogen, alkyl and —C(O)R¹⁰;
R⁶ is selected from the group consisting of hydrogen, alkyl and —C(O)R¹⁰;
R⁷ is selected from the group consisting of hydrogen, alkyl, aryl, heteroaryl, —C(O)R¹⁰ and —C(O)R¹⁷; or
R⁶ and R⁷ may combine to form a group selected from the group consisting of —(CH₂)₄—, —(CH₂)₅— and —(CH₂)₆—;
with the proviso that at least one of R⁵, R⁶ or R⁷ must be —C(O)R¹⁰;
R⁸ and R⁹ are independently selected from the group consisting of hydrogen, alkyl and aryl;
R¹⁰ is selected from the group consisting of hydroxy, alkoxy, aryloxy, —N(R¹¹)(CH₂)ₙR¹² and —NR¹³R¹⁴;
R¹¹ is selected from the group consisting of hydrogen and alkyl;
R¹² is selected from the group consisting of —NR¹³R¹⁴, hydroxy, —C(O)R¹⁵, aryl, heteroaryl, —N⁺(O⁻)R¹³R¹⁴, —N(OH)R¹³ and —NHC(O)R^{a} (wherein R^{a} is unsubstituted alkyl, haloalkyl or aralkyl);
R¹³ and R¹⁴ are independently selected from the group consisting of hydrogen, alkyl, cyanoalkyl, cycloalkyl, aryl and heteroaryl; or
R¹³ and R¹⁴ may combine to form a heterocycle group;
R¹⁵ is selected from the group consisting of hydrogen, alkoxy, hydroxy and aryloxy;
R¹⁶ is selected from the group consisting of hydroxy, —C(O)R¹⁵, —NR¹³R¹⁴ and —C(O)NR¹³R¹⁴;
R¹⁷ is selected from the group consisting of alkyl, cycloalkyl, aryl and heteroaryl;
R²⁰ is alkyl, aryl, aralkyl, heteroaryl or heteroaralkyl; and
n and r are independently 1, 2, 3 or 4;
or a salt such as a pharmaceutically acceptable salt thereof;
comprising the step of reacting a compound of formula (III): 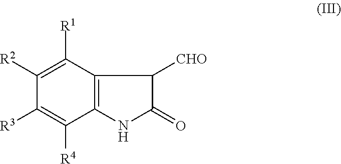
or a salt thereof, wherein R¹ to R⁴ are as hereinbefore described, with a compound of formula (II): 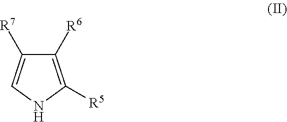
or a salt thereof, wherein R⁵ to R⁷ are as hereinbefore described.

60. The process according to claim 59, wherein compound (II) is a carboxylic acid having structure (IIa): 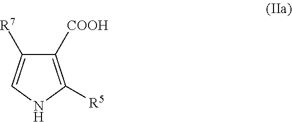
or a salt thereof, wherein R⁵ and R⁷ are as defined in claim 59; or wherein compound (II) is an amide having structure (IIb): 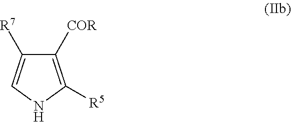
or a salt thereof, wherein:
R⁵ and R⁷ are as defined in claim 59;
R is selected from the group comprising —N(R¹¹)(CH₂)ₙR¹² and —NR¹³R¹⁴; and
R¹¹ to R¹⁴ and n are as defined in claim 59.

61. The process according to claim 59, for preparing sunitinib having structure: 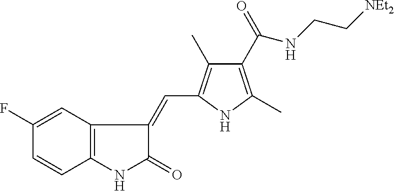
or a pharmaceutically acceptable salt thereof, the process comprising the step of reacting a compound of formula (IIc): 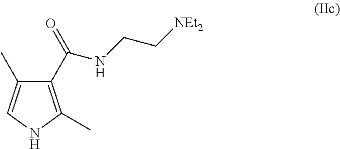
or a salt thereof, with a compound of formula (IIIa): 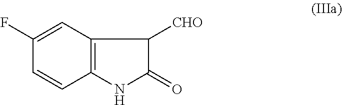
or a salt thereof.

62. The process according to claim 59, for preparing sunitinib having structure: 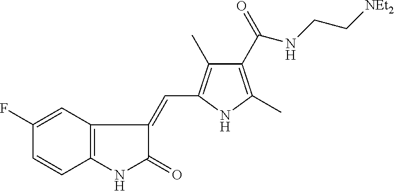
or a pharmaceutically acceptable salt thereof, the process comprising the steps of reacting a compound of formula (IIIa): 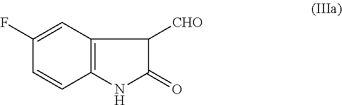
or a salt thereof, with a compound of formula (IIa) or (IIb): 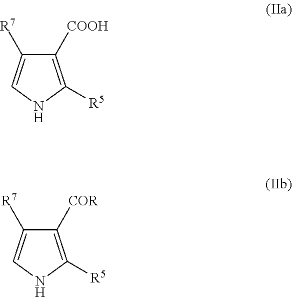
or a salt thereof, and optionally converting the resulting intermediate to sunitinib, wherein:
R⁵ and R⁷ are methyl;
R is selected from the group comprising —N(R¹¹)(CH₂)ₙR¹² and —NR¹³R¹⁴; and
R¹¹ to R¹⁴ and n are as defined in claim 59.

63. The process according to claim 59, wherein the reaction occurs in:
(i) an acidified polar solvent system; and/or
(ii) an acidified polar solvent system, wherein the polar solvent is a hydroxylic solvent; and/or
(iii) an acidified polar solvent system, wherein the polar solvent is ethanol; and/or
(iv) an acidified polar solvent system, wherein the acid is selected from the group comprising mineral acids or organic acids; and/or
(v) an acidified polar solvent system, wherein the acid is hydrochloric acid.

64. A process for preparing an acid of formula (IIa), (IIa′) or (IIa″): 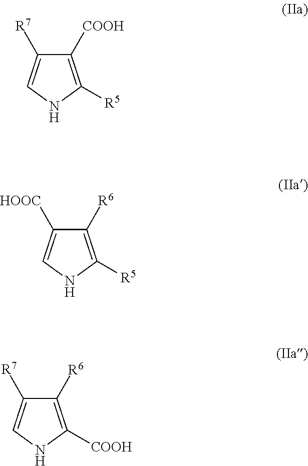
or a salt thereof, wherein said acid (IIa), (IIa′) or (IIa″) is formed from the corresponding pyrrole ester (IId), (IId′) or (IId″): 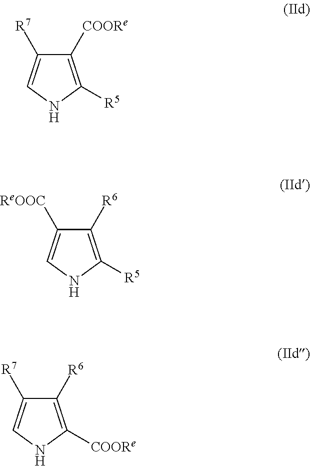
or a salt thereof, wherein:
R⁵ to R⁷ are as defined in claim 59; and
R^{e} is an alkyl, aryl, heteroaryl, aralkyl, cycloalkyl or heterocycle group.

65. The process according to claim 64, wherein the acid (IIa), (IIa′) or (IIa″) or a salt thereof is formed from the corresponding pyrrole ester (IId), (IId′) or (IId″) or a salt thereof by hydrolysis, optionally wherein the hydrolysis is performed in a solvent system comprising:
(i) one or more polar solvent(s) and a base; and/or
(ii) a combination of methanol, water and potassium hydroxide.

66. The process according to claim 64, wherein the pyrrole ester (IId) is a compound having structure (IIe): 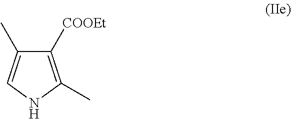
or a salt thereof.

67. A process for preparing an amide of formula (IIb), (IIb′) or (IIb″): 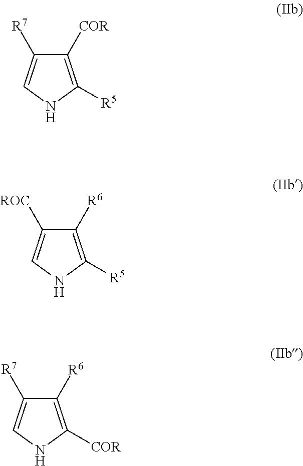
or a salt thereof, wherein said amide (IIb), (IIb′) or (IIb″) is formed from the corresponding acid (IIa), (IIa′) or (IIa″): 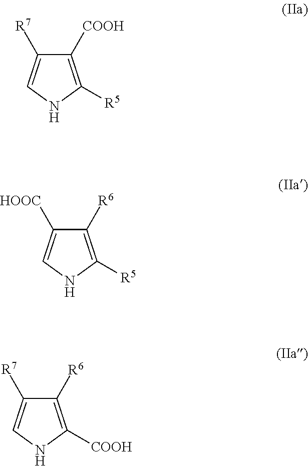
or a salt thereof, wherein:
R⁵ to R⁷ are as defined in claim 59; and
R is as defined in claim 60.

68. A process for preparing an amide of formula (Ib), (Ib′) or (Ib″): 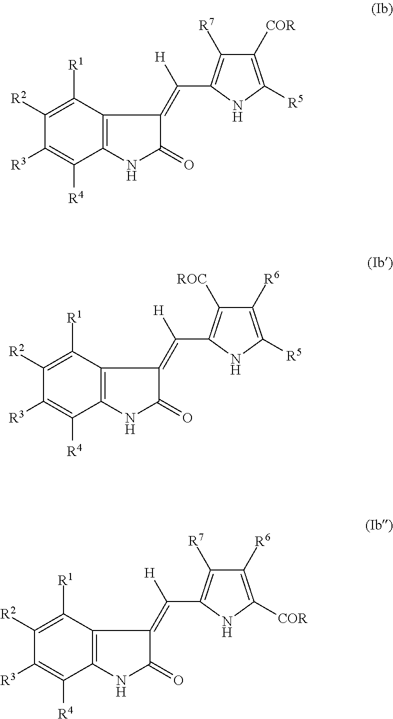
or a salt such as a pharmaceutically acceptable salt thereof, wherein said amide (Ib), (Ib′) or (Ib″) is formed from the corresponding acid (Ia), (Ia′) or (Ia″) 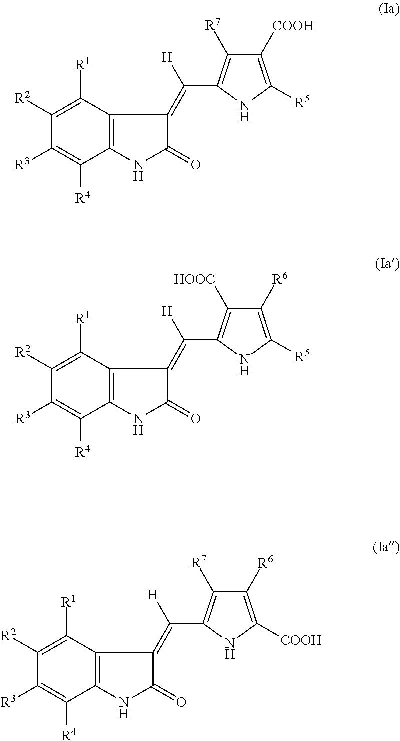
or a salt thereof, wherein:
R¹ to R⁷ are as defined in claim 59; and
R is as defined in claim 60.

69. The process according to claim 68, wherein said process is for preparing sunitinib having structure: 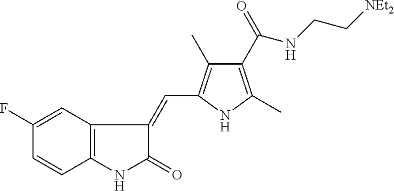
or a salt such as a pharmaceutically acceptable salt thereof, from the corresponding acid 5-[(5-fluoro-2-oxo-1,2-dihydro-3H-indol-3-ylidene)methyl]-2,4-dimethyl-1H-pyrrole-3-carboxylic acid: 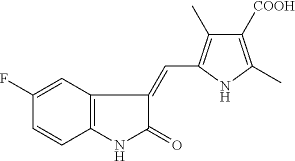
or a salt thereof.

70. The process according to claim 67, wherein the acid is converted to the corresponding amide via chemical activation of the —COOH group and subsequent reaction with RH or a salt thereof, and optionally wherein:
(i) the chemical activation is achieved via the use of a carbodiimide coupling reagent, optionally in conjunction with 1-hydroxybenzotriazole (HOBT) and/or a suitable base; and/or
(ii) the chemical activation is achieved via the use of 1-ethyl-3-(3-dimethylaminopropyl)carbodiimide hydrochloride, HOBT and triethylamine (TEA); and/or
(iii) RH is N,N-diethylethylenediamine or a salt thereof; and/or
(iv) the reaction is performed in a polar aprotic solvent; and/or
(v) the reaction is performed in THF.

71. The process according to claim 68, wherein the acid is converted to the corresponding amide via chemical activation of the —COOH group and subsequent reaction with RH or a salt thereof, and optionally wherein:
(i) the chemical activation is achieved via the use of a carbodiimide coupling reagent, optionally in conjunction with 1-hydroxybenzotriazole (HOBT) and/or a suitable base; and/or
(ii) the chemical activation is achieved via the use of 1-ethyl-3-(3-dimethylaminopropyl)carbodiimide hydrochloride, HOBT and triethylamine (TEA); and/or
(iii) RH is N,N-diethylethylenediamine or a salt thereof; and/or
(iv) the reaction is performed in a polar aprotic solvent; and/or
(v) the reaction is performed in THF.

72. A process for preparing a compound of formula (III): 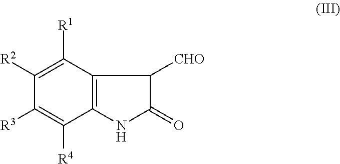
or a salt thereof, comprising adding a formyl group at the 3-position of a 2-oxindole having structure (IIIc) 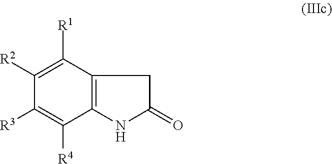
or a salt thereof, wherein R¹ to R⁴ are as defined in claim 59.

73. The process according to claim 72, for preparing a compound of formula (IIIa): 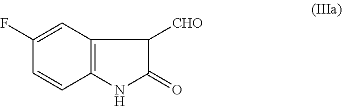
or a salt thereof, comprising adding a formyl group at the 3-position of a 2-oxindole having structure (IIIe): 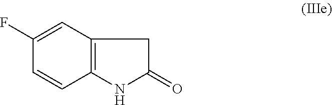
or a salt thereof.

74. The process according to claim 72:
(i) comprising reacting the 2-oxindole (IIIc) or (IIIe) or a salt thereof with ethyl formate; and/or
(ii) wherein the reaction takes place in the presence of a hydroxylic solvent and one of sodium methoxide, sodium ethoxide or sodium metal.

75. A process for preparing a 2-oxindole compound of formula (IIIc): 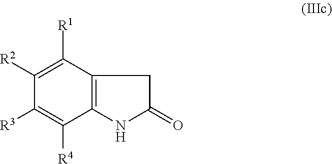
or a salt thereof, comprising reacting hydrazine hydrate with an isatin having structure (IIId) 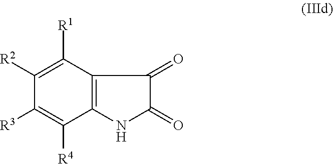
or a salt thereof, wherein R¹ to R⁴ are as defined in claim 59.

76. The process according to claim 75, for preparing a compound of formula (IIIe): 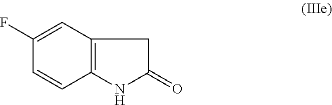
or a salt thereof, comprising reacting hydrazine hydrate with 5-fluoro-isatin having structure (IIIf): 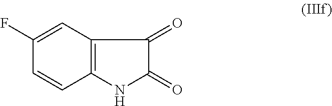
or a salt thereof.

77. The process according to claim 75, wherein:
(i) the reaction takes place in the presence of a hydroxylic solvent and one of sodium methoxide, sodium ethoxide or sodium metal; and/or
(ii) the reaction takes place in the presence of sodium methoxide; and/or
(iii) the isatin is added stepwise to the hydrazine hydrate.

78. A method comprising two or more processes selected from:
(a) the process according to claim 75 or any claim dependent thereon;
(b) the process according to claim 72 or any claim dependent thereon;
(c) the process according to claim 64 or any claim dependent thereon; and
(d) the process according to claim 59 or any claim dependent thereon.

79. The method according to claim 78, where the two or more processes may further be selected from:
(e) the process according to claim 68.

80. The method according to claim 78, where the two or more processes may further be selected from:
(f) the process according to claim 67.

81. The process according claim 59, for the preparation of:
(i) sunitinib and/or a salt, solvate or polymorph thereof; and/or
(ii) the malic acid salt of sunitinib; and/or
(iii) the L-malic acid salt of sunitinib.

82. A compound having structure III: 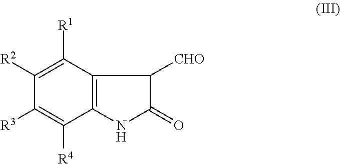
or a salt thereof, wherein R¹ to R⁴ are as defined in claim 59; preferably having structure (IIIa): 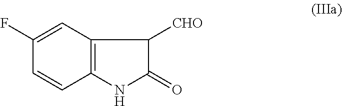
or a salt thereof.

83. A compound having structure (IIa), (IIa′) or (IIa″): 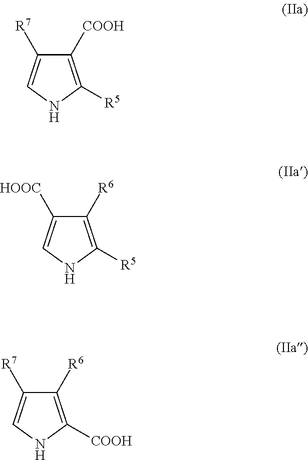
or a salt thereof, wherein R⁵ to R⁷ are as defined in claim 59; preferably having structure (IIf) 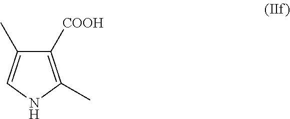
or a salt thereof.

84. A compound having structure (Ia): 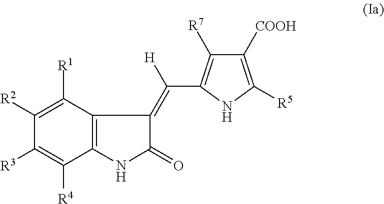
or a salt thereof, wherein:
R¹ to R⁴ are as defined in claim 59; and
R⁵ and R⁷ are each independently selected from hydrogen or alkyl;
preferably having structure: 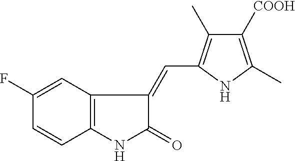
or a salt thereof.

85. A compound of formula (I) or a salt such as a pharmaceutically acceptable salt thereof prepared by a process according to claim 59.

86. A compound of formula (I) or a salt such as a pharmaceutically acceptable salt thereof prepared using an intermediate according to claim 82.

87. A compound of formula (I) or a salt such as a pharmaceutically acceptable salt thereof prepared using an intermediate according to claim 83.

88. A compound of formula (I) or a salt such as a pharmaceutically acceptable salt thereof prepared using an intermediate according to claim 84.

89. A pharmaceutical composition comprising a compound of claim 85 or a pharmaceutically acceptable salt thereof and one or more pharmaceutically acceptable excipient(s).

90. A pharmaceutical composition comprising a compound of claim 86 or a pharmaceutically acceptable salt thereof and one or more pharmaceutically acceptable excipient(s).

91. A pharmaceutical composition comprising a compound of claim 87 or a pharmaceutically acceptable salt thereof and one or more pharmaceutically acceptable excipient(s).

92. A pharmaceutical composition comprising a compound of claim 88 or a pharmaceutically acceptable salt thereof and one or more pharmaceutically acceptable excipient(s).

93. The composition according to claim 89, wherein:
(i) the compound is sunitinib malate; and/or
(ii) the composition is a tablet or a capsule.

94. The composition according to claim 90, wherein:
(i) the compound is sunitinib malate; and/or
(ii) the composition is a tablet or a capsule.

95. The composition according to claim 91, wherein:
(i) the compound is sunitinib malate; and/or
(ii) the composition is a tablet or a capsule.

96. The composition according to claim 92, wherein:
(i) the compound is sunitinib malate; and/or
(ii) the composition is a tablet or a capsule.

97. A method of treating a protein kinase mediated disorder, comprising administering to a patient in need thereof a therapeutically effective amount of a compound of claim 85 or a pharmaceutically acceptable salt thereof.

98. A method of treating a protein kinase mediated disorder, comprising administering to a patient in need thereof a therapeutically effective amount of a compound of claim 86 or a pharmaceutically acceptable salt thereof.

99. A method of treating a protein kinase mediated disorder, comprising administering to a patient in need thereof a therapeutically effective amount of a compound of claim 87 or a pharmaceutically acceptable salt thereof.

100. A method of treating a protein kinase mediated disorder, comprising administering to a patient in need thereof a therapeutically effective amount of a compound of claim 88 or a pharmaceutically acceptable salt thereof.
